SEQUENCE LISTING (13 sequences)

SEQ ID NO: 1 - RNA, 19 nt, Artificial Sequence
  MST1 antagonist - siRNA
uaaagagaccggccagauu

SEQ ID NO: 2 - RNA, 19 nt, Artificial Sequence
  MST1 antagonist - siRNA
gaugggcacuguccgagua

SEQ ID NO: 3 - RNA, 19 nt, Artificial Sequence
  MST1 antagonist - siRNA
gcccucauguagucaaaua

SEQ ID NO: 4 - RNA, 19 nt, Artificial Sequence
  MST1 antagonist - siRNA
ccagagcuauggucagaua

SEQ ID NO: 5 - protein, 11 aa, Artificial Sequence
  Potential MST1-targeted phosporylation site of PDX1 - T11
QYYAATQLYKD

SEQ ID NO: 6 - protein, 21 aa, Artificial Sequence
  Conserved phosporylation site in PDX1 - Human, Hamster and Horse
MNGEEQYYAATQLYKDPCAFQ

SEQ ID NO: 7 - protein, 11 aa, Artificial Sequence
  Potential MST1-targeted phosporylation site of PDX1 - T126
PWMKSTKAHAW

SEQ ID NO: 8 - protein, 11 aa, Artificial Sequence
  Potential MST1-targeted phosporylation site of PDX1 - T152
NKRTRTAYTRA

SEQ ID NO: 9 - protein, 11 aa, Artificial Sequence
  Potential MST1-targeted phosporylation site of PDX1 - T155
TRTAYTRAQLL

SEQ ID NO: 10 - protein, 11 aa, Artificial Sequence
  Potential MST1-targeted phosporylation site of PDX1 - T214
KRGGGTAVGGG

SEQ ID NO: 11 - protein, 11 aa, Artificial Sequence
  Potential MST1-targeted phosporylation site of PDX1 - T231
QDCAVTSGEEL

SEQ ID NO: 12 - protein, 21 aa, Artificial Sequence
  Conserved phosporylation site in PDX1 - Mouse and Rat
MNSEEQYYAATQLYKDPCAFQ

SEQ ID NO: 13 - protein, 21 aa, Artificial Sequence
  Conserved phosporylation site in PDX1 - Dog
MNSEEQFYAATQLYKDPCAFQ

(19) United States
(12) Patent Application Publication
(10) Pub. No.: US 2016/0083733 A1
(43) Pub. Date: Mar. 24, 2016

(54) NOVEL TREATMENT OF METABOLIC DISEASES

(71) Applicant: UNIVERSITY OF BREMEN, Bremen (DE)

(72) Inventors: Kathrin MAEDLER, Bremen (DE); Amin ARDESTANI, Bremen (DE)

(21) Appl. No.: 14/892,729

(22) PCT Filed: May 23, 2014
(86) PCT No.: PCT/EP2014/060678

§ 371 Date: Nov. 20, 2015

(30) Foreign Application Priority Data
May 23, 2013 (EP) 13168987.9

(51) Int. Cl. C12N 15/113; A61K 38/17; C07K 16/40; A61K 38/08

(52) U.S. Cl. CPC C12N 15/1137; A61K 38/08; A61K 38/1709; C07K 16/40; C12N 2310/14; C12N 2310/531; C07K 2317/21; C07K 2317/76

(57) ABSTRACT

Antagonists of Mammalian Sterile 20-like kinase (MST) 1 for use in the treatment and prevention of metabolic diseases, in particular diabetes and obesity are described.

### FIELD OF THE INVENTION

The present invention generally relates to antagonists of Mammalian Sterile 20-like kinase 1 (MST1) for use in the treatment and prevention of metabolic diseases, in particular diabetes and obesity. In addition the present invention relates to compositions comprising an MST1 antagonist or an MST1 antagonist and an anti-diabetic and/or an anti-obesity related disease agent.

### BACKGROUND OF THE INVENTION

Metabolism is the process the body uses to get or make energy from food. Food is made up of proteins, carbohydrates, and fats. In the digestive system food parts are broken down into sugars and acids, i.e. the body's fuel which the body can use right away to produce and consume energy, or store in the body tissues, such as liver, muscles, and body fat. A metabolic disorder occurs when abnormal chemical reactions in the body disrupt this process leading to too much of some substances or too little of other ones that are needed to stay healthy. A metabolic disease or disorder may develop when some organs, such as the liver or pancreas, become diseased or do not function normally. One of the most prominent examples of a metabolic disease is diabetes.

Diabetes is a metabolic disease in which the body is unable to produce sufficient amounts of insulin to maintain normoglycemia. Diabetes was reported by Greek physicians already 250 B.C. and is the Greek word for “syphon”, referring to the severe condition of polyuria, the production of large amounts of urine. The complete term “diabetes mellitus” was established later in the 17th century. Mellitus is Latin for honey, which is how the physician Thomas Willis described the taste of urine in patients.

Blood glucose levels are controlled by pancreatic hormones produced by different cell types within the organized structures of the islets of Langerhans that form the endocrine portion of the pancreas. In particular the hormone insulin, produced by the β-cells, is responsible for decreasing blood glucose by inducing its uptake into target tissues after meals. Diabetes manifests when β-cells fail to produce sufficient amounts of insulin, due to a loss of function and the loss of β-cells themselves. A number of studies over the years, either performed on mouse models or by investigating autopsy material from human pancreata show that a hallmark of diabetes in both autoimmune type 1 diabetes (T1D) as well as obesity related type 2 diabetes (T2D) is the loss of insulin producing β-cells by apoptosis (Kurrer et al., PNAS 94 (1997), 213-218; Donath et al., J. Mol. Med. 81 (2003), 455-470; Butler et al., Diabetes 52 (2003), 102-110).

Current therapies for the treatment of diabetes are directed towards alleviating the symptoms of the disease, but there is an urgent medical need for therapies that slow or prevent the onset of the disease and preferably are capable of reconstituting the insulin-producing β-cells and β-cell function, respectively, and restoring insulin secretion.

The solution to the technical problem is achieved by providing the embodiments as characterized in the claims and described further below.

### SUMMARY OF THE INVENTION

The present invention generally relates to therapeutic compounds capable of modulating mammalian sterile 20-like kinase 1 (MST1) activity for use in the treatment of metabolic diseases, in particular diabetes and obesity or obesity-related diseases, in particular if they are associated with diabetes. Typically, such modulators in accordance with the present invention are MST1 antagonists capable of for example inhibiting MST1 kinase activity or reducing the level of active MST1.

In one embodiment of the present invention, the MST1 antagonist is for use in the treatment of diabetes including the treatment and prevention of type 1 diabetes (T1D), type 2 diabetes (T2D), progressive hyperglycemia and/or improving glucose tolerance.

In a further embodiment, the present invention relates to compositions comprising an MST1 antagonist or an MST1 antagonist and a further therapeutic agent, preferably an anti-diabetic agent and/or anti-obesity related disease agent.

In still a further embodiment, the present invention relates to an MST1 antagonist which is an anti-diabetic agent and/or an anti-diabetic agent comprises an MST1 antagonist.

In another embodiment, the present invention relates to a dietary food product comprising an MST1 antagonist either alone or in combination with a further anti-diabetic agent and/or anti-obesity agent.

In yet another embodiment, the present invention provides a non-human animal which is genetically engineered either transient or stably to exhibit a reduced level of MST1 activity compared to a corresponding wild-type (WT) animal, which reduced level of MST1 activity is pancreatic β-cell specific. Preferably, such a non-human transgenic animal in accordance with the present invention is a transgenic β-cell specific MST1^{-/-} knock-out mouse.

In a still further embodiment, the present invention provides novel MST1 antagonists such as derived from β-cell transcription factor PDX1 (pancreatic duodenal homeobox 1), for example peptide kinase inhibitors and PDX1 variants which are inert to phosphorylation by MST1 at amino acid site threonine (Thr) 11. Otherwise Thr11 phosphorylation results in PDX1 ubiquination and degradation and subsequent reduction in PDX1 target genes and loss of glucose-stimulated insulin secretion as well as β-cell death.

Further embodiments of the present invention will be apparent from the description and Examples that follow.

### BRIEF DESCRIPTION OF THE DRAWINGS

FIG. 1: Characterization of β-cell specific MST1^{-/-} mice. 2 month-old (n=8) β-MST1^{-/-} mice and fl/fl controls (n=6) were examined. (a) Western blot analysis of protein lysates from the islets of β-MST1^{-/-} and Pip-Cre control mice. (b) Intraperitoneal glucose tolerance test (ipGTT) with 1 g/kg body weight glucose. (c) Intraperitoneal insulin tolerance test (ipITT) with 0.75 IU/kg body weight insulin.

FIG. 2: β-cell specific inhibition of MST1 activity by β-cell specific disruption of MST1 prevents hyperglycemia and diabetes progression. (a-h) β-MST1^{-/-} mice with specific deletion in the β-cells using the Cre-Lox system (n=5) and their Rip-Cre (n=3) and fl/fl controls (n=3) were injected with 40 mg/kg streptozotocin for 5 consecutive days. (a) Random fed blood glucose measurements after last STZ injection (day 0) over 32 days. (b) Intraperitoneal glucose tolerance test (ipGTT) performed after 12 h fast with 1 g/kg body weight glucose. (c, d) Insulin secretion during an ipGTT measured before (0 min) and 30 min after glucose injection and data are expressed as (d) ratio of secreted insulin at 30 min/0 min (stimulatory index). (e) The ratio of secreted insulin and glucose is calculated at fed state. (f-h) Mice were sacrificed at day 32. (f) β-cell mass per pancreas on fixed, paraffin embedded sections calculated as the product of the relative cross-sectional area of β-cells (determined by quantification of the cross-sectional area occupied by β-cells divided by the cross-sectional area of total tissue multiplied with the weight of the pancreas). 10 sections per mouse spanning the width of the pancreas were included in the analysis. (g, h) Triple staining for (g) TUNEL or (h) Ki67, insulin and DAPI performed. Results are expressed as percentage of (g) TUNEL- or (h) Ki67-positive β-cells±SE. Data show mean±SE. *p<0.05 β-MST-STZ compared to fl/fl-STZ or Cre-STZ mice.

FIG. 3: MST1 signaling pathway in regulating pancreatic β-cells and apoptosis. Based on the results obtained in vivo with the mouse models described in Examples 1 and 2 and FIGS. 1 to 4 and the experiments with MST1/PDX1 interaction under diabetic conditions and in particular with Thr11A mutant PDX1 described in Example 5 and FIGS. 17 to 20 a reliable complex picture of MST1 signaling pathway could be established taking into account previous preliminary in vitro data. Diabetic stimuli lead to activation of MST1. Active MST1 triggers cytochrome c release and mitochondrial-dependent apoptosis by modulating Bim/Bax/Bcl2/Bcl-xL through JNK/AKT signaling. Active caspase-9 then triggers cleavage of caspase-3, which triggers the caspase-3-dependent cleavage of MST1 to its constitutively active fragment, which leads to further MST1 activation and processing of caspase-3 by a positive feedback mechanism, and acceleration of β-cell death occurs. Cleaved MST1 translocates to the nucleus and directly phosphorylates PDX1 (it may not be excluded the possibility that MST1 targets PDX1 also in cytoplasm) and histone H2B. PDX1 then shuttles to cytosol, where it marks for ubiquitination and subsequent degradation by proteasome machinery and β-cell function is impaired. Histone H2B phosphorylation by MST1 also induces chromatin condensation, one of the characteristic features of apoptosis. Importantly, due to the identification of the target phosphorylation site at Thr11 of PDX1 and in particular demonstrating that mutation thereof antagonizes MST1 mediated impairment of β-cells it is now feasible to develop and employ MST1 based anti-diabetic agents blocking the MST1/PDX1 signaling pathway only, for example by using mutant Thr11A PDX1 or a peptide comprising the phosphorylation site while the MST1 apoptotic pathway may remain unaffected in kind.

FIG. 4: Inhibition of MST1 activity by MST1 deletion protects from diabetes in vivo. (a-l) MST1^{-/-} mice (n=15) and their littermates (n=14) were injected with 40 mg/kg streptozotocin or citrate buffer for 5 consecutive days. (a) Random fed blood glucose measurements after last STZ injection (day 0) over 21 days. (b) Intraperitoneal glucose tolerance test (ipGTT) performed at day 17 after 12 h fast with 1 g/kg body weight glucose. (c, d) Insulin secretion during an ipGTT measured before (0 min) and 30 min after glucose injection and data are expressed as (d) ratio of secreted insulin at 30 min/0 min (stimulatory index). (e) The ratio of secreted insulin and glucose is calculated at fed state. (f-l) Mice were sacrificed at day 22. (f) β-cell mass per pancreas on fixed, paraffin embedded sections calculated as the product of the relative cross-sectional area of β-cells (determined by quantification of the cross-sectional area occupied by β-cells divided by the cross-sectional area of total tissue multiplied with the weight of the pancreas). 10 sections per mouse spanning the width of the pancreas were included in the analysis. (g, h) Triple staining for (g) TUNEL or (h) Ki67, insulin and DAPI performed. Results are expressed as percentage of (g) TUNEL- or (h) Ki67-positive β-cells±SE. The mean number of β-cells scored was 23121 for each treatment condition. (i, j) The pancreatic area of β- (stained in red; j) and β-cells (stained in green; j) are given as percentage of the whole pancreatic section from 10 sections spanning the width of the pancreas. (k, l) Representative double-staining for Bim (red, k) or PDX1 (red, l) and insulin (green) is shown from STZ-treated MST1^{-/-} mice and controls. White arrows indicate areas of cytosolic PDX1 localization and its total absence in WT-STZ mice. (m-s) MST1^{-/-} (n=5) and WT (n=6) mice were fed a high fat/high sucrose diet (HFD) for 16 weeks and thereafter injected with a single dose of STZ (100 mg/kg) and kept for 3 more weeks under HFD treatment. (m) ipGTT with 1 g/kg body weight glucose. (n, o) Insulin secretion during an ipGTT measured before (0 min) and 30 min after glucose injection, (o) data are expressed as ratio of secreted insulin at 30 min/0 min (stimulatory index). (p) The ratio of secreted insulin and glucose is calculated at fed state. (q) β-cell mass was analyzed as described above (r, s) Triple staining for TUNEL (r) or Ki67 (s) in red, insulin in green and DAPI in blue. Results are expressed as percentage of (r) TUNEL- and (s) Ki67-positive β-cells±SE. The mean number of β-cells scored was 25639 for each treatment condition. *p<0.05 WT-STZ compared to WT saline injected mice, **p<0.05 MST1⁴⁻-STZ compared to WT-STZ mice. +MST1^{-/-} compared to WT littermates.

FIG. 5: Inhibition of MST1 activity by MST1 deletion protects from HFD/STZ-induced diabetes. Whole body MST1^{-/-} (n=5) and WT (n=6) mice were fed a high fat/high sucrose diet (HFD) for 16 weeks and thereafter injected with a single dose of STZ (100 mg/kg) and kept for 3 more weeks under HFD treatment. (a,b) Intraperitoneal insulin tolerance tests (ipITT) with 0.75 IU/kg body weight insulin, (b) the difference of the highest (0 min) and lowest (60 min) glucose concentration was calculated. (c-e) 10 fixed, paraffin embedded pancreas sections per mouse spanning the width of the pancreas were stained for insulin and (c) percentage of β-cell fraction of the whole pancreas (d) islet density/cm² pancreas and (e) mean islet size analyzed using NIS-elements microscopical analysis software. (f, g) Triple staining for TUNEL (f) or Ki67 (g) in red, insulin in green and DAPI in blue.

FIG. 6: MST1 deletion has no effect on glycemia nor insulin secretion. 2 month- (a-d; n=9) and 6 month-old (e-h; n=5) MST1^{-/-} mice and their littermates (n=5) were examined. (a, e) Intraperitoneal glucose tolerance tests (ipGTT) with 1 g/kg body weight glucose. (b, f) Intraperitoneal insulin tolerance tests (ipITT) with 0.75 IU/kg body weight insulin. (c, d, g, h) Insulin secretion during an ipGTT measured before (0 min) and 30 min after glucose injection and data are expressed as (d, h) ratio of secreted insulin at 30 min/0 min (stimulatory index).

FIG. 7: MST1 deletion protects from diabetes. (a-f) MST1^{-/-} mice (n=15) and their littermates (n=14) were injected with 40 mg/kg streptozotocin or citrate buffer for 5 consecutive days and sacrificed at day 22. (a-c) 10 fixed, paraffin embedded pancreas sections per mouse spanning the width of the pancreas were stained for insulin and (a) percentage of β-cells of the whole pancreas (b) islet density/cm² pancreas and (c) mean islet size analyzed using NIS-elements microscopic analysis software. (d, e) Triple staining for TUNEL (d) or Ki67 (e) in red, insulin in green and DAPI in blue performed on pancreatic section from different mice groups. (f) Representative double-staining for GLUT2 (red) and insulin (green) is shown from the different groups. (g, h) MST1 deficiency rescued from STZ-induced apoptosis in vitro. (g) Isolated islets from MST1^{-/-} and control mice or (h) stable INS-1E shMST1 and shScr clones were exposed to 1 mM STZ for 6 h. (g) β-cell apoptosis was analyzed by double staining of TUNEL and insulin. Results are expressed as percentage of TUNEL-positive β-cells±SE from 3 independent experiments. The mean number of β-cells scored was 6776 for each treatment condition. (h) P-MST1, Bim, caspase-3 and PARP cleavage were analyzed by western blotting. Western blot shows representative results from 3 independent experiments. Tubulin was used as loading control. *p<0.05 STZ treated compared to vehicle treated control, **p<0.05 MST1^{-/-} compared to WT at same treatment.

FIG. 8: MST1 induces β-cell death through activation of the mitochondrial apoptotic pathway. (a, b) Human islets left untreated or infected with Ad-GFP or Ad-MST1 for 48 h. β-cell apoptosis was analyzed by triple staining for DAPI (blue), TUNEL (red) and insulin (green; a). An average number of 18501 insulin-positive β-cells were counted in 3 independent experiments from 3 different donors (b). (c-i) Adenovirus-mediated MST1 or GFP (control) overexpression in human islets and INS-1E cells. (c, d) Efficient up-regulation of MST1 is achieved by adenoviral system. Profiling expression levels of proteins of the mitochondrial death pathway showed up-regulation of Bim, Bax and caspase-9 cleavage as well as down-regulation of Bcl-2 and Bcl-xL together with JNK activation and caspase-3 and PARP cleavage upon MST1 overexpression in human islets and INS-1E cells. (e) Exposure of Ad-GFP- or Ad-MST1-infected human islets to Bax-inhibitory peptide V5 or negative control (NC) peptide for 36 h. Caspase-3 cleavage was analyzed by western blotting (f, g). Human islets transfected with Bim siRNA or control siScr were infected with Ad-GFP or Ad-MST1 for 48 h. (f) β-cell apoptosis was analyzed by double staining of TUNEL and insulin. An average number of 10,378 insulin-positive β-cells were counted in 3 independent experiments from 3 different donors. (g) Bim, caspase-3 and PARP cleavage were analyzed by western blotting. (h) Human islets were transfected with GFP or a dominant negative mutant of JNK1 (dnJNK1) expressing-plasmids and infected with Ad-GFP or Ad-MST1 for 48 h. P-C-Jun, Bim and caspase-3 cleavage were analyzed by western blotting. (i) Human islets were transfected with GFP or Myr-AKT1 expressing-plasmids and infected with Ad-GFP or Ad-MST1 for 48 h. Bim and caspase-3 cleavage were analyzed by western blotting. All western blots show representative results from at least 3 independent experiments from 3 different donors (human islets). Tubulin/Actin was used as loading control. Results shown are means±SE. *p<0.05 MST-OE compared to GFP control, **p<0.05 siBim-MST1 compared to siScr-MST1.

FIG. 9: MST1 is activated by diabetogenic conditions and correlates with β-cell apoptosis. (a-d) Activated MST1 (cleaved and phosphorylated) in human and mouse islets and INS1-E cells. (a) Human islets, (b) mouse islets and (c,d) INS-1E cells exposed to diabetogenic conditions (22.2-33.3 mM glucose or the mixture of 33.3 mM glucose and 0.5 mM palmitate (33.3 Palm) or IL-1β/IFNγ (ILIF) for 72 h. MST1, P-MST1, P-JNK, P-H2B and caspase-3 cleavage were analyzed by western blotting. (e-h) Activated MST1 in diabetic islets. (e) Human isolated islets from non-diabetic controls and patients with T2D, all with documented fasting plasma glucose >150 mg/dl and (f) from 10-week old diabetic db/db and their heterozygous db/+ littermates were cultured for 24 h after isolation and MST1 activity analyzed by western blotting. (g, h) Double immunostaining for P-MST1 in red and insulin in green in sections from human isolated islets from non-diabetic controls and patients with T2D and from 6-week old diabetic db/db mice (magnification ×200). (i) INS-1E cells transfected with GFP control or Myr-AKT1 expression-plasmids and exposed to 33.3 mM glucose for 72 h. (j, k) PI3K/AKT was inhibited in INS-1E cells by exposure to (j) PI3K inhibitor, LY294002 (10 μM for 8 h) or (k) AKT inhibitor Triciribine (10 μM for 6 h). (l) INS-1E infected with Ad-GFP or Ad-MST1 or transfected with shMST1 or shScr control expression plasmids. 48 h after infection/transfection, cells were serum-starved for 12 h and then stimulated by adding 100 nM insulin for 15 min. MST1, P-MST1, P-AKT, P-GSK3, P-FOXO1 and caspase-3 cleavage were analyzed by western blotting. All western blots show representative results from at least 3 independent experiments from 3 different donors or mice. Tubulin/Actin was used as loading control. (e-h) Representative analyses from 10 pancreata from patients with T2D and >10 controls and from 7 db/db and 7 db/+ controls are shown.

FIG. 10: MST1 induces β-cell apoptosis through the mitochondrial apoptotic pathway. (a) Analysis of mitochondrial pathway of cell death in Ad-MST1 infected human islets. (b) cytochrome c release in INS-1E cells. COX was used to confirm a clean mitochondrial fraction. (c) INS-1E cells were infected by Ad-GFP or Ad-MST1 and exposed to 33.3 mM glucose for 48 h. MST1, P-MST1, Bim, caspase-3 and PARP cleavage were analyzed by western blotting. All western blots show representative results from 3 independent experiments (c: blots representative from 2 independent experiments) from 3 donors (human islets). Actin and tubulin were used as loading control.

FIG. 11: JNK mediates MST1-induced Bim induction and apoptosis. Human islets were pretreated with JNK selective inhibitor, SP600125 (25 μM) or vehicle control for 1 h and infected by Ad-GFP or Ad-MST1 for 48 h. P-C-Jun, Bim and caspase-3 cleavage were analyzed by western blotting. The western blot shows representative results from 3 independent experiments from 3 different donors. Actin was used as loading control.

FIG. 12: Diabetogenic conditions induce MST1 activation. (a) Human islets and (b) INS-1E cells exposed to diabetogenic conditions (33.3 mM glucose, 0.5 mM palmitate or the mixture of 33.3 mM glucose and 0.5 mM palmitate (33.3 Palm) for 72 h (human islets) and 24 h (INS-1E cells) or 100 μM H₂O₂ for 6 h). (c) Isolated islets from normal diet (ND) or high fat/high sucrose (HFD)-fed mice treated for 16 weeks. MST1, P-MST1 and caspase-3 cleavage were analyzed by western blotting. All western blots show representative results from 3 independent experiments from 3 different donors or mice. Actin was used as loading control.

FIG. 13: MST1 deficiency improves β-cell survival and function. (a-d) Human islets transfected with MST1 siRNA (smart pool, mixture of 4 siRNA) or control siScr and were treated with the cytokines mixture IL/IF, 33.3 mM glucose or the mixture of 33.3 mM glucose and 0.5 mM palmitate (33.3 Palm) for 72 h. (a) β-cell apoptosis was analyzed by double staining of TUNEL and insulin. An average number of 11390 insulin-positive β-cells were counted for each treatment condition in 3 independent experiments from 3 different donors. (b) Western blotting confirmed successful (˜80%) MST1 depletion in human islets. MST1, P-MST1, Bim, caspase-9 and -3 cleavage and P-H2B all were analyzed by western blotting. (c) RT-PCR for BCL2L11 was performed in human islets and levels normalized to tubulin shown as change from siScr control transfected islets. (d) Insulin stimulatory index denotes the ratio of secreted insulin during 1 h-incubation with 16.7 mM and 1 h-incubation with 2.8 mM glucose. (e, f) Islets were isolated from MST1^{-/-} mice and their WT littermates and exposed to the cytokines mixture IL/IF or the mixture of 33.3 mM glucose and 0.5 mM palmitate (33.3 Palm) for 72 hours. (e) β-cell apoptosis was analyzed by double staining for TUNEL and insulin. An average number of 24180 insulin-positive β-cells were counted for each treatment condition in 3 independent experiments. (f) Insulin stimulatory index denotes the ratio of secreted insulin during 1 h-incubation with 16.7 mM and 1 h-incubation with 2.8 mM glucose. (g-i) Stable INS-1E clones were generated by transfection of vectors for shMST1 and shScr control and treated with the cytokines mixture IL/IF or 33.3 mM glucose for 72 h. (g) MST1, Bim, PDX1, caspase-3 and PARP cleavage were analyzed by western blotting (h) Insulin stimulatory index denotes the ratio of secreted insulin during 1 h-incubation with 16.7 mM and 1 h-incubation with 2.8 mM glucose. (i) PDX1 target genes in shMST1 and shScr control INS-1E cells exposed to 5.5 or 33.3 mM glucose for 72 h were analyzed by RT-PCR and levels normalized to tubulin and shown as change from shScr control INS1-E clones. Western blots (b, g) show representative results from 3 independent experiments from 3 different donors (human islets). Tubulin/Actin was used as loading control. TUNEL data (a, e), GSIS (d, f, h) or RT-PCR (c, i) show pooled results from 3 independent experiments. Results shown are means±SE. *p<0.05 compared to siScr (a, c, d), WT (e, f) or shScr untreated controls (h, i), **p<0.05 compared to siScr (a, c, d), WT (e, f) or shScr (h, i) at the same treatment conditions.

FIG. 14: MST1 inhibition preserves β-cell survival and function in vitro. (a) Human islets transfected with MST1 siRNA (smart pool, mixture of 4 siRNA) or control siScr were treated with H₂O₂ for 6 h. P-MST1, Bim and caspase-3 cleavage were analyzed by western blotting. (b-e) Stable INS-1E shMST1 and shScr clones were treated with diabetogenic conditions (b: 0.5 mM palmitate for 72 h, c: 100 μM H₂O₂ for 6 h, d: cytokine mix IL-1β/IFNγ for 72 h or e: 33.3 mM glucose). (b,c) P-MST1, caspase-3 and PARP cleavage were analyzed by western blotting. (d,e) Cytochrome c release from mitochondria to cytosol was analyzed. Cytochrome c, COX and tubulin were analyzed by western blotting. (f) INS-1E cells were transfected with GFP control or do-MST1 (K59) plasmids and treated with 33.3 mM glucose for 48 h. MST1, caspase-3 and PARP cleavage were analyzed by western blotting. (g) Glucose stimulated insulin secretion during 1 h-incubation with 2.8 mM and 16.7 mM glucose, respectively, normalized to insulin content in MST1-depleted INS-1E cells exposed to IL-1β/IFNγ or 33.3 mM glucose for 72 h. Western blots show representative results from 3 independent experiments from 3 different donors (human islets). Tubulin/Actin was used as loading control. GSIS (g) show pooled results from 3 independent experiments.

FIG. 15: MST1 impairs β-cell function through destabilization of PDX1. (a-d) Adenovirus-mediated GFP or MST1 overexpression in human islets for 96 h. (a-b) MST1 overexpression abolished glucose-induced insulin secretion. (a) Insulin secretion during 1 h-incubation with 2.8 mM (basal) and 16.7 mM glucose (stimulated). (b) The insulin stimulatory index denotes the ratio of secreted insulin during 1 h-incubation with 16.7 mM and 2.8 mM glucose, respectively. (c) MST1 and PDX1 immunoreactivity were analyzed by Western blotting. (d) PDX1 target genes including SLC2A2, GCK and Insulin were analyzed by RT-PCR. (e-g) HEK293 cells were transfected with plasmids encoding Myc-MST1 and GFP-PDX1. (e) A kinase-dead MST1 (dn-MST1: K59R) was co-transfected with GFP-PDX1. (f) At 48 h after transfection, HEK293 cells were treated with 50 μg/ml cycloheximide (CHX) for 8 h. (g) At 36 h after transfection, HEK293 cells were treated with the proteasome inhibitor MG-132 (50 μM) for 6 h. PDX1 and MST1 were analyzed by western blotting. (h, i) In vivo ubiquitination assay in (h) HEK293 cells and (i) human islets. (h) HEK293 cells were transfected with GFP-PDX1 and HA-ubiquitin, alone or together with Myc-MST1 or MST1-K59 expression plasmids for 48 h. (i) Human islets (2 different donors) were transfected with HA-ubiquitin and infected with Ad-GFP or Ad-MST1 for 48 h. MG-132 was added during the last 6 h of the experiment. HEK293 or islets lysates were immunoprecipitated with an anti-PDX1 antibody followed by immunoblotting with ubiquitin antibody to detect ubiquitinated PDX1. (j) HEK293 cells were transfected with GFP-PDX1 alone or together with Myc-MST1 for 48 h. Reciprocal co-immunoprecipitations performed using anti-GFP and anti-Myc antibodies and western blot analysis performed with precipitates and input fraction using anti-Myc and anti-GFP antibodies, respectively. (k) In vitro kinase assay was performed by incubating recombinant MST1 and PDX1 proteins and analyzed by NuPAGE followed by western blotting using pan-phospho-threonine specific, PDX1 and MST1 antibodies. (l) Lysates of HEK293 cells transfected with PDX1-WT or PDX1-T11A expression-plasmids were immunoprecipitated with PDX1 antibody and subjected to an in vitro kinase assay using recombinant MST1. Phosphorylation reactions were analyzed by Western blotting using p-T11-PDX1 specific and pan-phospho threonine antibodies. (m) HEK293 cells were transfected with PDX1-WT or PDX1-T11A alone or together with MST1 expression-plasmids for 48 h. MST1 and PDX1 were analyzed by western blotting. (n) PDX-1-WT or PDX1-T11A was co-transfected with MST1 in HEK293 cells. At 36 h after transfection, cells were treated with 50 μg/ml CHX for the times indicated, and lysates subjected to western blotting with PDX1 antibody and densitometry analysis of bands performed. (o) PDX1 overexpression was shown by transfecting human islets with GFP control, PDX1-WT or PDX1-T11 expressing plasmids. PDX1 was analyzed by western blotting. (p, q) Human islets were transfected with PDX1-WT or PDX1-T11A expression-plasmids and infected with Ad-GFP or Ad-MST1 for 72 h. (p) Insulin stimulatory index denotes the ratio of secreted insulin during 1 h-incubation with 16.7 mM and 2.8 mM glucose, respectively. (q) PDX1 target genes in human islets analyzed by RT-PCR and levels normalized to tubulin and shown as change from PDX1-WT transfected islets. All western blots show representative results from at least 3 independent experiments from 3 different donors (human islets). Tubulin/Actin was used as loading control. RT-PCR (d, q) and GSIS (b, p) show pooled results from 3 independent experiments from 3 different donors. Results shown are means±SE. *p<0.05 MST-OE compared to GFP (b, d, p, q) control, **p<0.05 compared to PDX-1WT-MST1.

FIG. 16: MST1 impairs β-cell function through PDX1 degradation. (a-d) INS-1E cells were infected with Ad-GFP or Ad-MST1 for 96 h. (a) Insulin secretion during 1 h-incubation with 2.8 mM (basal) and 16.7 mM and glucose (stimulated) (b) Insulin stimulatory index denotes the ratio of secreted insulin during 1 h-incubation with 16.7 mM and 2.8 mM glucose. (c) MST1 and PDX1 were analyzed by western blotting in INS-1E cells. (d) PDX1 target genes including SLC2A2, GCK, Ins1 and Ins2 were analyzed by RT-PCR in INS-1E cells. (e, f) Luciferase reporter assay. (e) HEK293 cells were transfected with PDX1, Ins2-Luc renilla and pCMV-firefly plasmids alone or together with MST1 for 48 h. (f) INS-1E cells transfected with Ins2-Luc renila and pCMV-firefly plasmids and infected with Ad-GFP or Ad-MST1 for 48 h. Data are expressed as RLU (renilla/firefly) normalized to controls. The western blot (c) shows representative results from 3 independent experiments. Actin was used as loading control. All other results are shown as means±SE from 3 independent experiments. *p<0.05 MST-OE compared to control.

FIG. 17: MST1 destabilizes PDX1 protein in human islets. Human islets were infected with Ad-GFP or Ad-MST1. 48 h after infection, islets were treated with 50 μg/ml cycloheximide (CHX) for 8 h. PDX1 was analyzed by western blotting. The western blot shows representative results from 3 independent experiments from 3 different donors. Tubulin was used as loading

FIG. 18: A diabetogenic milieu increases the PDX1-MST1 interaction. INS1E cells exposed to 11.1 mM glucose control with or without IL/IF or 33.3 mM glucose for 72 h. Lysates of INS1 cells were immunoprecipitated with PDX1 and IgG control antibodies, followed by immunoblotting for MST1 and PDX1. Representative results from 2 independent experiments are shown.

FIG. 19: MST1 phosphorylates PDX1 in vitro and in vivo. (a) Purified human recombinant MST1 and PDX1 proteins were incubated with ³²P-labeled ATP for 30 min at 30° C. Reactions were analyzed by NuPAGE followed by autoradiography. (b) Lysates of HEK293 cells transfected with GFP-PDX1 alone or together with Myc-MST1 expression plasmids were immunoprecipitated with PDX1 antibody. Immunoprecipitation and input fractions were analyzed by NuPAGE followed by western blotting using pan-phospho threonine specific, PDX1 and MST1 antibodies. Representative results from 3 independent experiments are shown.

FIG. 20: MST1 specifically phosphorylates PDX1 on Thr11 site. (a) Potential theoretical PDX1 phosphorylation sites by MST1 were predicted by Netphos 2.0 program. (b) The six candidate sites of phosphorylation by MST1 were individually mutated to alanine to generate phospho-deficient mutants. In vitro kinase assay was performed by incubating recombinant PDX1-GST fusion proteins including different mutants of PDX1 (purified from bacteria) and MST1. Reaction was analyzed by NuPAGE followed by western blotting using pan-phospho threonine specific and PDX1 antibodies. (c) Western blot analysis of in vitro kinase reaction using phospho-specific antibody generated against phosphorylated Thr11 form of PDX1 (pT11-PDX1). (d) In vivo kinase assay. Lysates of HEK293 cells transfected with PDX1-WT or PDX1-T11A alone or together with Myc-MST1 expressing plasmids, were immunoprecipitated with PDX1 antibody. IP reaction was analyzed by NuPAGE followed by western blotting using pan-phospho threonine, pT11-PDX1 and PDX1 antibodies. (e) Alignment of the conserved phosphorylation site in PDX1 (Thr11, red) from different species.

FIG. 21: Thr11 mutation stabilizes PDX1 and preserves β-cell function. (a) In vitro ubiquitination assay. HEK293 cells were transfected with PDX1-WT or PDX1-T11A together with Myc-MST1 and HA-ubiquitin expression plasmids for 48 h and MG-132 was added during the last 6 h of the experiment. Lysates were immunoprecipitated by PDX1 antibody followed by immunoblotting with ubiquitin antibody to detect ubiquitinated PDX1. (b) Luciferase reporter assay. HEK293 cells were transfected with Ins2-Luc renila, pCMV-firefly, PDX1-WT or PDX1-T11A, alone or together with Myc-MST1 expressing plasmids for 48 h. The data expressed as RLU (renilla/firefly) normalized to the PDX1-WT. (c, d) INS-1E were transfected with PDX1-WT or PDX1-T11A expression-plasmids and infected with Ad-GFP or Ad-MST1 for 72 h. (c) Insulin stimulatory index denotes the ratio of secreted insulin during 1 h-incubation with 16.7 mM and 1 h-incubation with 2.8 mM glucose. (d) PDX1 target genes in INS-1E cells analyzed by RT-PCR and levels normalized to tubulin shown as change from PDX1-WT transfected INS-1E cells. (a, c) Representative results from 2 independent experiments are shown. All other results (b, d) are shown as means±SE from 3 independent experiments. *p<0.05 compared to control. **p<0.05 compared to PDX1-WT-MST1.

FIG. 22: JNK and caspase-3 are responsible for stress-induced MST1 cleavage and apoptosis. (a, b) Human islets and INS-1E were pretreated with JNK selective inhibitor, SP600125 (25 μM for human islets, 10 μM for INS-1E cells) or vehicle control for 1 h and then exposed to diabetogenic conditions (33.3 mM glucose or IL-1β/IFNγ) for 72 h. (c) Human islets transfected with caspase-3 siRNA or control siScr and treated with IL/IF for 72 h. (d) INS-1E cells were pretreated with pan-caspase inhibitor z-DEVD-fmk (50 μM; Caspi) or vehicle control for 1 h and then exposed to ER-stress inducer thapsigargin (1 μM) for 6 h. MST1, P-C-Jun, caspase-3 and PARP cleavage were analyzed by western blotting. All western blots show representative results from 2 independent experiments from 2 donors (human islets). Tubulin/Actin was used as loading control.

FIG. 23: MST1-AKT crosstalk: AKT suppresses MST1 activation and β-cell apoptosis. (a-c) INS-1E cells pretreated with (a, c) GLP1 (100 nM) or (b) insulin (100 nM) with or without PI3K inhibitor LY294002 (10 μM) for 1 h were exposed to diabetogenic conditions (a, b: IL-1β/IFNγ or c: 33.3 mM glucose) for 72 h. P-AKT, MST1 and caspase-3 cleavage were analyzed by western blotting. (d) INS-1E cells transfected with GFP control or Myr-AKT1 expression-plasmids and exposed to IL-1β/IFNγ for 72 h. MST1, P-MST1, P-AKT, P-GSK3 and caspase-3 cleavage were analyzed by western blotting. All western blots show representative results from 2 independent experiments. Actin was used as loading control.

FIG. 24: MST1-AKT crosstalk. AKT inhibition induces MST1 activation and β-cell apoptosis. (a) AKT was inhibited in human islets by exposure to AKT inhibitor Triciribine (20 μM for 24 h). P-AKT, P-GSK3, MST1 and caspase-3 cleavage were analyzed by western blotting. (b) Human islets and INS-1E cells were transfected with siRNA against Akt1/2/3 and siScr control and treated with IL/IF for 72 h. T-AKT, MST1 and caspase-3 cleavage were analyzed by western blotting. (c, d) Stable INS-1E shMST1 and shScr clones were treated with AKT inhibitor (c; 10 μM for 6 h) or LY294002 (d; 10 μM for 8 h). Caspase-3 cleavage was analyzed by western blotting. All western blots show representative results from 2 independent experiments from 2 donors (human islets). Actin was used as loading control.

FIG. 25: MST1-AKT crosstalk: AKT inhibition induces MST1 activation and β-cell apoptosis. (a) AKT was inhibited in human islets by exposure to AKT inhibitor Triciribine (20 μM for 24 h). P-AKT, P-GSK3, MST1 and caspase-3 cleavage were analyzed by western blotting. (b) Human islets and INS-1E cells were transfected with siRNA against Akt1/2/3 and siScr control and treated with IL/IF for 72 h. T-AKT, MST1 and caspase-3 cleavage were analyzed by western blotting. (c, d) Stable INS-1E shMST1 and shScr clones were treated with AKT inhibitor (c; 10 μM for 6 h) or LY294002 (d; 10 μM for 8 h). Caspase-3 cleavage was analyzed by western blotting. All western blots show representative results from 2 independent experiments from 2 donors (human islets). Actin was used as loading control.

### DETAILED DESCRIPTION OF THE INVENTION

The present invention generally relates to therapeutic compounds agents capable of modulating mammalian sterile 20-like kinase 1 (MST1) activity for use in the treatment of metabolic diseases and disorders. Typically, such modulators in accordance with the invention are MST1 antagonists capable for example inhibiting MST1 kinase activity or reducing the level of active MST1. Unless indicated otherwise the terms “substance”, “compound” and “agent” are used interchangeably herein and include but are not limited to, nucleic acids (e.g., DNA and RNA), carbohydrates, lipids, proteins, peptides, antibodies, peptidomimetics, small molecules and other drugs and pro-drugs; for substances, compounds and agents which may be used in accordance with the present inventions see also US patent application US 2004/0213794 A1, the disclosure content of which is incorporated herein by reference, in particular paragraphs [0122] to [0131]. In accordance with the present invention the term “substance”, “compound” and “agent” also relates to means which are not compounds in the classical sense, for example radiation, stress such as heat and chilling, culture conditions, and the like which result directly or indirectly in substantially reducing MST1 kinase activity or the level of active MST1 or nullify it altogether such as observed in MST1 knock-out mice.

The present invention is based on the surprising finding that MST1 deficiency restored β-cell function and survival in diabetic animal models. In particular, ablation of MST1 protected mice from β-cell failure and the development of diabetes induced either by multiple low dose-STZ (T1D model) or high-fat diet (T2D model); this protective action was due to an inhibition of apoptosis, enhanced proliferation, normalized a- and β-cell ratio and restored β-cell mass. Strikingly, β-cell-specific disruption of MST1 expression also prevented progressive hyperglycemia and improved glucose tolerance in MLD-STZ-treated mice indicating that β-cell-specific activation of MST1 is a key event in the progressive loss of β-cells in diabetes. These findings identify MST1 as novel pro-apoptotic kinase and key mediator of the apoptotic signaling cascade in the β-cell and thus as a new target for the therapeutic intervention in the treatment of diabetes and related metabolic diseases. Thus, in its broadest aspect the present invention relates to a Mammalian Sterile 20-like kinase 1 (MST1) antagonist for use in the treatment of a metabolic disease.

As used herein, the term “metabolic disease” refers to disorders of metabolic processes, see also the background section, supra, and may be accompanied by one or more of the following symptoms: an increase in visceral obesity, serum glucose, and insulin levels, along with hypertension and dyslipidemia. It can be congenital due to inherited enzyme abnormality or acquired due to disease of an endocrine organ or failure of a metabolically important organ such as the pancreas. Within the term metabolic disease the term “metabolic syndrome” is a name for a group of symptoms that occur together and are associated with the increased risk of developing coronary artery disease, stroke, and T2D. The symptoms of metabolic syndrome include central or abdominal obesity, high blood pressure, high triglycerides, insulin resistance, low HDL cholesterol, and tissue damage caused by high glucose.

MST1 (EC 2.7.11; Seq.: Chromosome 20; NC_{—}000020.10) also known as STK4, KRS2 is an ubiquitously expressed serine/threonine kinase. The nucleotide and amino acid sequences of MST1 can be retrieved form publicly available databases, for example at EMBL under accession number BC005231 or GenBank under accession number NG_{—}032172.1. The nucleotide and amino acid sequences of MST1 and its structural characterization are also described in international application WO 99/15635, the disclosure content of which is incorporated herein by reference, in particular with respect to the nucleotide and amino acid sequences of human MST1.

MST1 it is part of the Hippo signaling pathway and involved in multiple cellular processes such as morphogenesis, proliferation, stress response and apoptosis (Ling et al., Cell Signal 20 (2008), 1237-1247; Avruch et al., Cell Dev. Biol. 23 (1998), 770-784). In addition to its pro-apoptotic function, MST1 has been shown to play an important role in tumorigenesis (Lu et al., PNAS 107 (2010), 1437-42; Song et al., PNAS 107 (2010), 1431-6; Zhou et al., Cancer Cell 16 (2009), 425-38). Loss or reduction of MST1 expression has also been correlated with pure cancer prognosis (Seidel et al., Mol. Carcinog. 46 (2007), 865-71). Furthermore, recent genetic studies have indicated that liver-specific deletion of MST1 and its closest paralog MST2 in mice resulted in liver enlargement, cancer, and resistance to TNF-induced apoptosis (Lu et al., PNAS 107 (2010), 1437-42; Song et al., PNAS 107 (2010), 1431-6; Zhou et al., Cancer Cell 16 (2009), 425-38). MST1 is a target as well as an activator of caspases to amplify the apoptotic signaling pathway (Lee et al., Oncogene 16 (1998), 3029-3037; Kakeya et al., Cancer Res. 58 (1998), 4888-4894). MST1 promotes cell death through regulation of multiple downstream targets such as LATS1/2, histone H2B, FOXO family members as well as induction of stress kinase c-Jun-N-terminal Kinase (JNK) and caspase-3 activation (Avruch et al., Cell Dev. Biol. 23 (1998), 770-784); Bi et al., J. Biol. Chem. 285 (2010), 6259-6264); Cheung et al., Cell 113 (2003), 507-517).

In international application WO 2012/121992 homozygous deficiency of the MST1 gene in mice has been described to significantly delay the onset of and alleviated the severity of Experimental Autoimmune Encephalitis (EAE). Furthermore, an MST1-deficient Collagen Induced Arthritis (CIA) mouse model is described to display a markedly decreased incidence of arthritis when compared with their WT littermates. However, hitherto a pancreatic β-cell specific deletion of MST1 had not been considered.

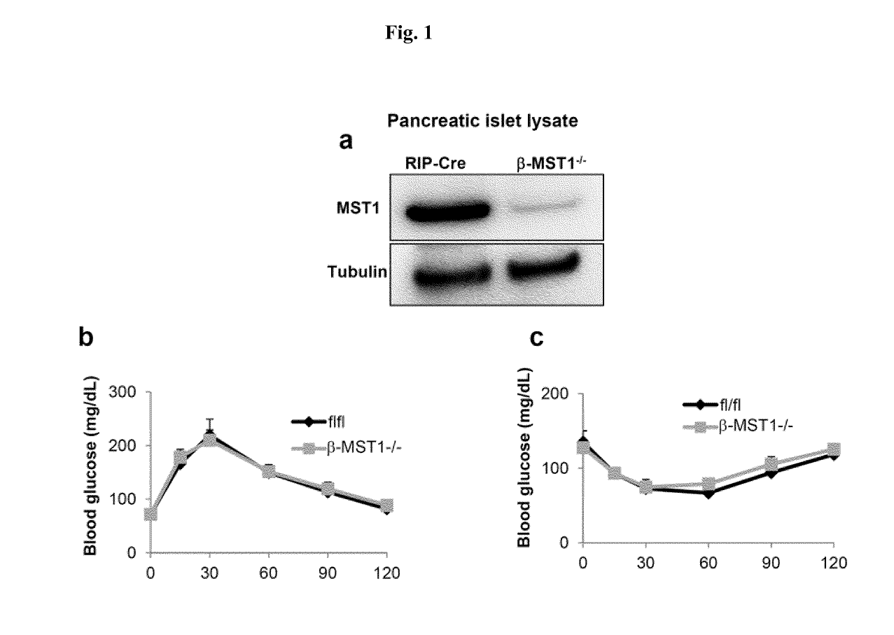

As demonstrated in the Examples, it was surprisingly found in accordance with the present invention that pancreatic β-cell specific disruption of MST1 prevents diabetes progression and hyperglycemia. In particular, in accordance with the present invention a pancreatic β-cell specific MST1 knock-out (β-MST1^{-/-}) mouse has been genetically engineered containing a null mutation for MST1, as confirmed by western blotting of lysates from isolated islets; see Example 1 and FIG. 1 a. The β-MST1^{-/-} mice were viable, fertile and showed no difference in food intake and body weight, glucose tolerance and insulin sensitivity compared to MST1fl/fl or flox-negative littermates (RIP-Cre); see Example 1 and FIG. 1 b, c.

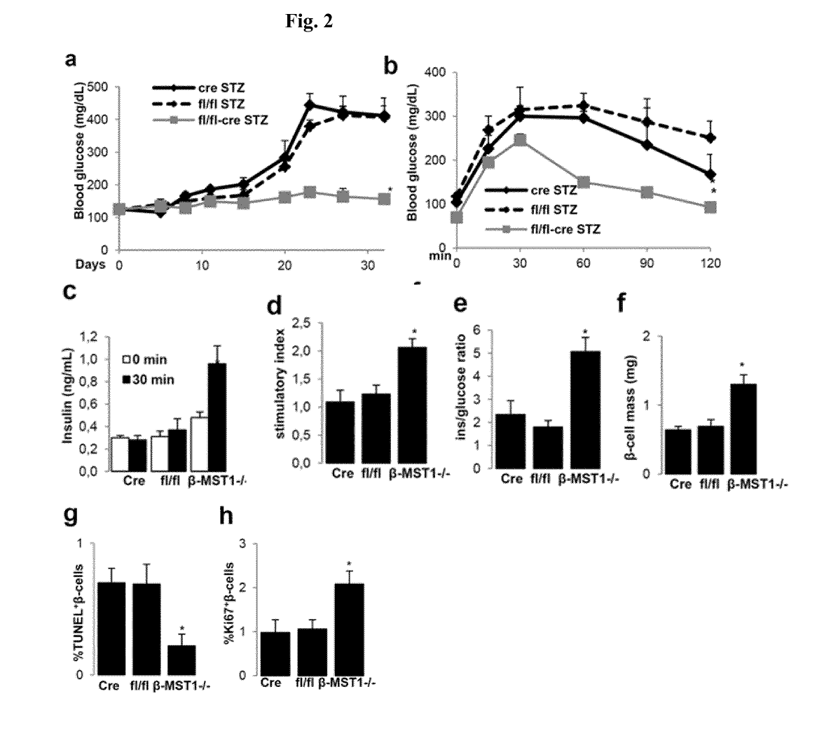

As shown in the Examples, β-MST1^{-/-} mice were protected against diabetes as assessed by multiple low-dose streptozotocin (MLD-STZ) injections (T1D model) and in comparison to RIP-Cre control mice (FIG. 2). After MLD-STZ treatment, blood glucose levels in MST1fl/fl and RIP-Cre control mice increased gradually (FIG. 2 a). While both control groups became overtly diabetic, reaching blood glucose levels >400 mg/dl, β-MST1^{-/-} mice maintained normal blood glucose levels (FIG. 2 a). Notably, MST1fl/fl and RIP-Cre control mice exhibited impaired glucose tolerance; this was strikingly improved in β-MST1^{-/-} mice (FIG. 2 b). This protection was accompanied by significant restoration of glucose-induced insulin response (FIG. 2 c, d) and insulin/glucose ratio (FIG. 2 e). β-cell protection was also confirmed by the significantly higher β-cell mass in the MLD-STZ β-MST1^{-/-} mice (FIG. 2 f) resulting from enhanced β-cell survival (FIG. 2 g) and proliferation (FIG. 2 h), compared to MST1fl/fl and RIP-Cre control mice. These data indicate that β-cell specific disruption of MST1 prevented progressive hyperglycemia and improved glucose tolerance in MLD-STZ-treated β-MST1^{-/-} mice as a result of decreased apoptosis and restoration of β-cell mass showing that β-cell specific activation of MST1 is a key event in the progressive loss of β-cells in diabetes.

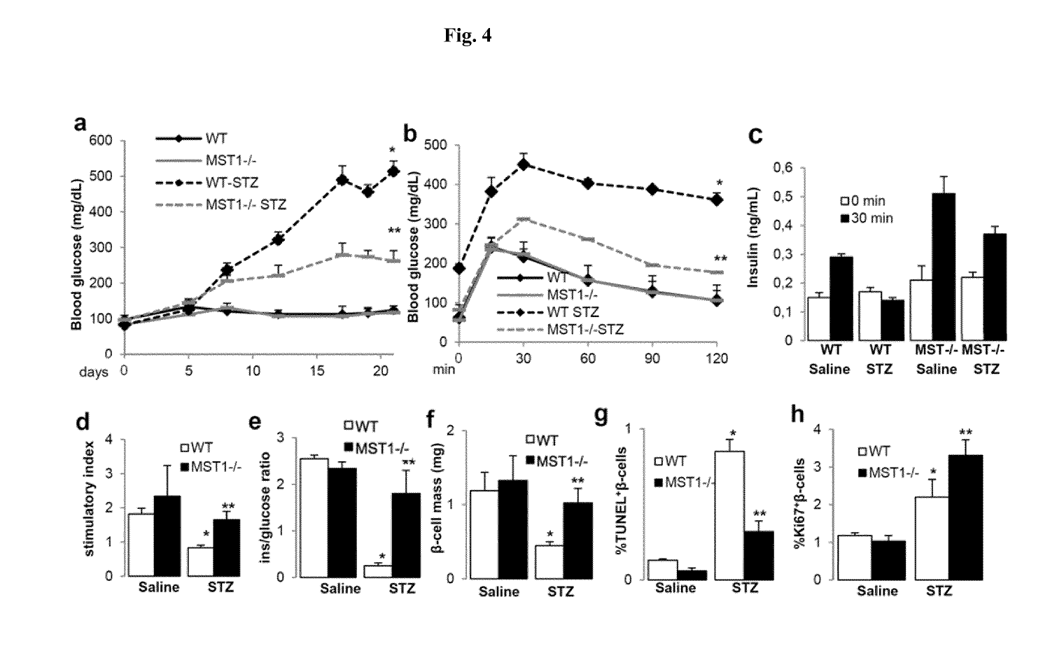

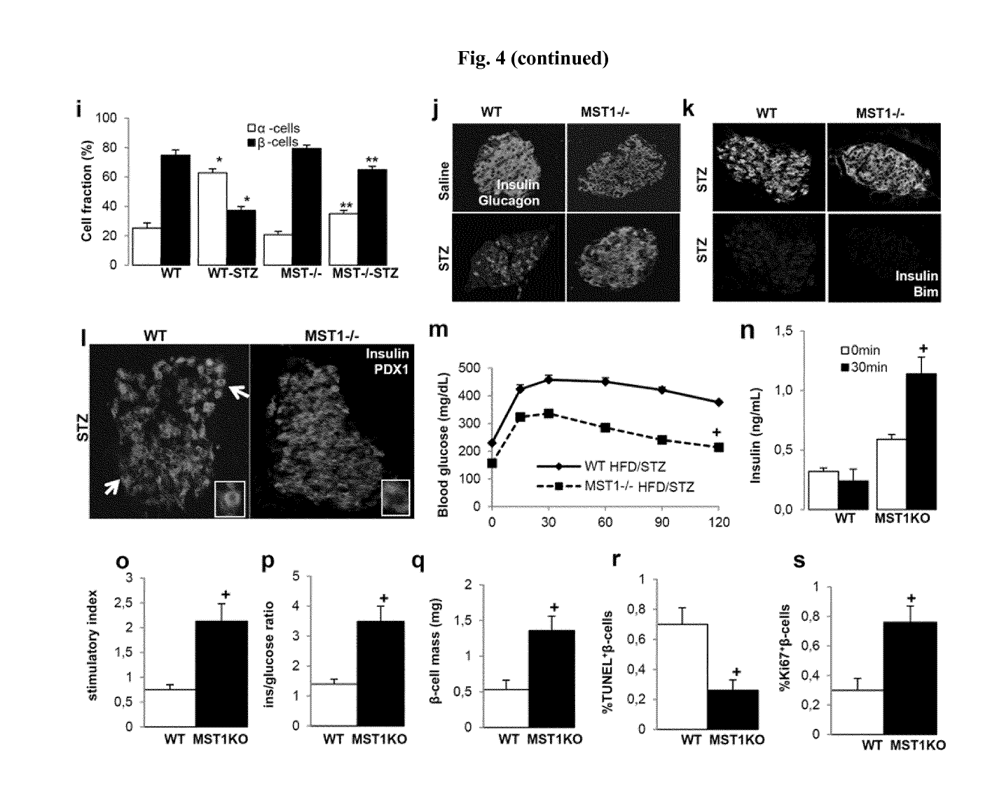

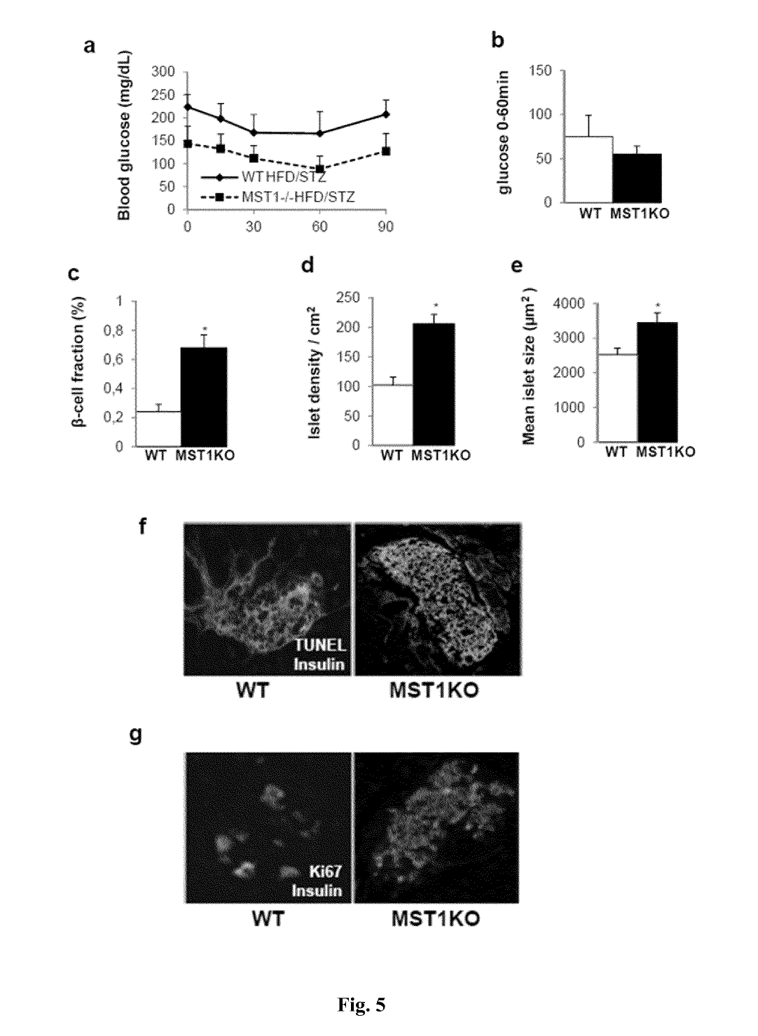

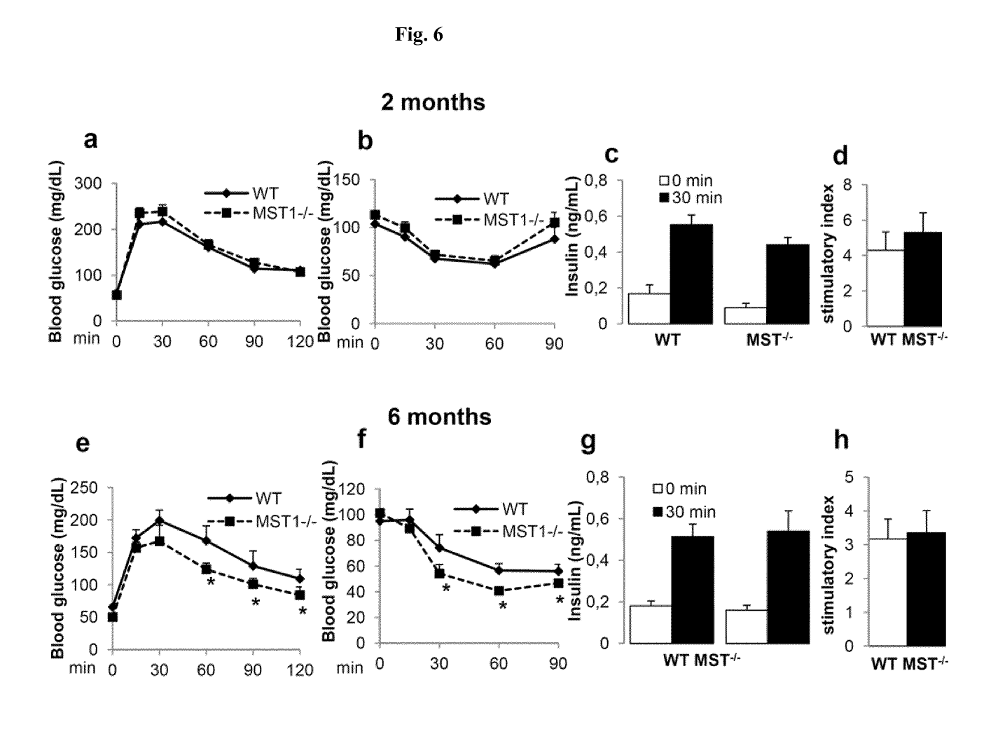

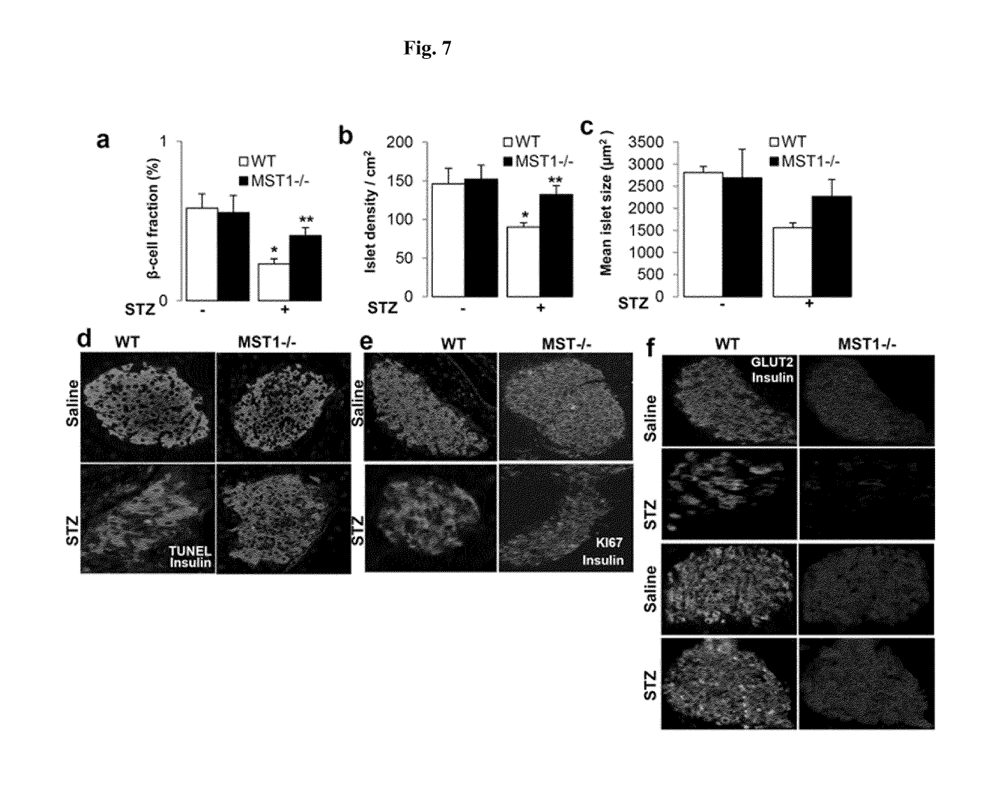

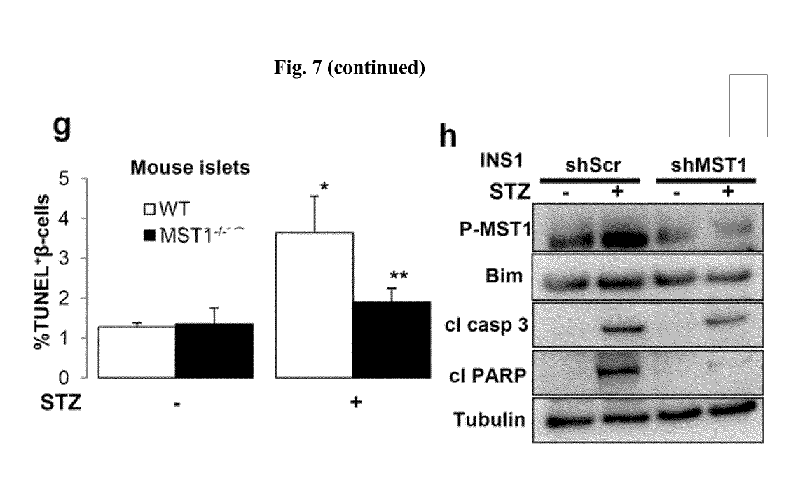

Additionally, the protective effect of inhibiting MST1 activity against hyperglycemia and development of diabetes was further confirmed in vivo in a high-fat diet (HFD) model (T2D). The combination of high fat-sucrose diet for 16 weeks and administration of a single dose of STZ (100 mg/kg) led to severe hyperglycemia 3 weeks after administration of STZ and impaired glucose tolerance in WT mice. Similar to the effects in the MLD-STZ model, inhibition of MST1 activity by MST1 deletion also resulted in improved glucose tolerance and insulin secretion, increased β-cell mass and restored β-cell morphology as a result of improved β-cell survival and proliferation, compared to HFD/STZ-treated littermate MST1^{+/+} mice (FIGS. 4 and 5). An intraperitoneal insulin tolerance test (ipITT) revealed that the glucose-lowering effects in the MST1^{-/-} mice can be accounted to the improved β-cell mass and insulin secretion, since MST1^{-/-} mice and their WT littermates showed similar insulin sensitivity (FIG. 5 a, b). The combination of these morphological, histochemical and metabolic data provide evidence that normalization of blood glucose and insulin concentrations, islet architecture, and β-cell mass by inhibition of MST1 activity such as by MST1 deletion after a diabetes-inducing injury occurs as a result of increased β-cell survival and proliferation.

The experiments performed in accordance with the present invention described in the appended Examples and illustrated in the Figures have meanwhile published by the inventors in the renowned journal Nat. Med. 20 (2014), 385-397; see for technical details the section “Methods and any associated references” that are available in the online version of the paper and the section “RESULTS” including the Figures and the Supplementary Figures as well as the legends thereto referred to therein in Ardestani et al., Nat. Med. 20 (2014), 385-397, the disclosure content of which is incorporated herein by reference in its entirety.

As is evident from the above, the pancreatic β-cell specific MST1 knock-out animal model illustrated in Example 1 is useful for the investigation of the mechanisms underlying metabolic diseases, in particular diabetes and obesity. Therefore, in a further aspect, the present invention relates to a non-human animal which is genetically engineered to exhibit a reduced level of MST1 activity compared to a corresponding wild-type (WT) animal, which reduced level of MST1 activity is pancreatic β-cell specific. Preferably, the non-human transgenic animal is a MST1 knock out animal. In a particular preferred embodiment, the animal is a rodent, preferably a mouse. Means and methods for generating transgenic animals are known to the person skilled in the art; see, e.g., Advanced Protocols for Animal Transgenesis, An ISTT Manual in the Series: Springer Protocols Handbooks Pease, Shirley; Saunders, Thomas L. (Eds.) 2011, XV ISBN 978-3-642-20792-1. Specific transgene expression in mouse pancreatic beta-cells under the control of the porcine insulin promoter is described in Grzech et al., Mol. Cell Endocrinol. 315 (2010), 219-224. Likewise, the rat insulin 2 gene (Ins2) promoter, widely used to achieve transgene expression in pancreatic beta-cells of mice, also directs expression to extrapancreatic tissues and performs poorly in isolated pancreatic islets of human, mouse, and pig. Alterations of Pancreatic Beta-cell Mass and Islet Number due to Ins2-controlled Expression of Cre Recombinase: RIP-Cre is reviewed in Pomplun et al., Horm. Metab. Res. 39 (2007), 1-5.

According to the WHO the term “diabetes” and “diabetes mellitus”, respectively, describes a metabolic disorder of multiple aetiology characterized by chronic hyperglycaemia with disturbances of carbohydrate, fat and protein metabolism resulting from defects in insulin secretion, insulin action, or both. The effects of diabetes mellitus include long-term damage, dysfunction and failure of various organs (WHO 1999).

As mentioned in the background section, there are two main types of diabetes; T1D usually develops in childhood and adolescence and patients require lifelong insulin injections for survival. T2D usually develops in adulthood and is related to obesity, lack of physical activity, and unhealthy diets. This is the more common type of diabetes (representing 90% of diabetic cases worldwide) and treatment may involve lifestyle changes and weight loss alone, or oral medications or even insulin injections.

Other categories of diabetes include gestational diabetes (a state of hyperglycemia which develops during pregnancy) and “other” rarer causes (genetic syndromes, acquired processes such as pancreatitis, diseases such as cystic fibrosis, exposure to certain drugs, viruses, and unknown causes). As well, intermediate states of hyperglycemia (impaired fasting glucose or impaired glucose tolerance) have been defined. These states are significant in that they can progress to diabetes, but with weight loss and lifestyle changes, this progression can be prevented or delayed.

In the short term, hyperglycemia causes symptoms of increased thirst, increased urination, increased hunger, and weight loss. However, in the long-term, it causes damage to eyes (leading to blindness), kidneys (leading to renal failure), and nerves (leading to impotence and foot disorders/possibly amputation). As well, it increases the risk of heart disease, stroke, and insufficiency in blood flow to legs. Studies have shown that good metabolic control prevents or delays these complications.

Thus, the primary goal of treatment is to bring the elevated blood sugars down to a normal range, both to improve symptoms of diabetes as well as to prevent or delay diabetic complications. In accordance with the present invention this goal is achieved by blocking MST1 for example by inhibitors of MST1 kinase activity, which as demonstrated in the Examples 1-3 directly restores survival of β-cells and improves glucose stimulated insulin secretion.

Accordingly, in a preferred embodiment of the present invention, the MST1 antagonist is for use in the treatment of all classifications of diabetes, preferably type 1 diabetes (T1D) or type 2 diabetes (T2D) or for preventing progressive hyperglycemia and/or improving glucose tolerance.

In T1D, autoimmune destruction of insulin-producing β-cells and critically diminished β-cell mass are hallmarks of the disease (Mathis et al., Nature 414 (2001), 792-798). β-cell destruction occurs through immune mediated processes; mononuclear cell infiltration in the pancreatic islets and interaction between antigen presenting cells and T-cells leads to high local concentrations of proinflammatory cytokines, e.g. interleukin (IL)-1 β, tumor necrosis factor (TNF) and interferon (IFN)-β, chemokines, reactive oxygen species (ROS) and other apoptotic triggers (e.g. the perforin and Fas/FasL system) (Thomas et al., Cell Death Differ 17 (2010), 577-585).

In contrast, in T2D β-cell dysfunction and reduced β-cell mass are the ultimate events leading to the development of clinically overt disease in insulin resistant patients. β-cell destruction is caused by multiple stimuli including glucotoxicity, lipotoxicity, pro-inflammatory cytokines, endoplasmatic reticulum and oxidative stress (Donath et al., J. Mol. Med. 81 (2003), 455-470; Potout et al., Endocr. Rev. 29 (2008), 351-366).

As shown in the Examples antagonizing MST1 leads to the inhibition of β-cell apoptosis, enhanced β-cell proliferation, normalized α- and β-cell ratio and restores β-cell mass. Therefore, in accordance with the present invention blocking MST1 and thus the use an MST1 antagonist is particularly advantageous in the treatment and prevention of metabolic syndrome including but not limited to T2D, abdominal obesity, high cholesterol and high blood pressure. Thus, in a particularly preferred embodiment of the present invention, the MST1 antagonist is for use in the treatment or prevention of type 2 diabetes (T2D), obesity, progressive hyperglycemia and/or for improving glucose tolerance. Preferably, the treatment an MST1 antagonist in accordance with the present invention is accompanied with restoration of β-cell survival and/or insulin secretion.

In principle, the MST1 antagonist for use in accordance with the present invention can be any compound or measure such as radiation or heat treatment which reduces level of MST1 and MST1 activity, disrupts MST1 signal pathway and/or counteracts MST1 activity; see also the Examples and supra. Unless indicated otherwise the term “antagonist” and “inhibitor” are used interchangeably herein and includes but is not limited to any nucleic acid, formulation, compound or substance that can regulate MST1 activity in such a way that MST1 is decreased or wherein the effects of MST1 are blocked or altered. Examples of MST1 antagonists include but are not limited to antibody, siRNA, shRNA, kinase inhibitor or a dominant mutant of MST1 (dnMST1). Anti-MST1 monoclonal antibodies are commercially available; see, e.g., MST1 monoclonal antibody (M04), clone 3B5 from Abnova, Catalog # H00004485-M04, Abnova GmbH c/o EMBLEM, Heidelberg, Germany. In case of mouse or rat monoclonal antibodies they may of course be humanized for the purpose of treating humans. Anti-MST1 siRNA are described in Example 3.

The term antagonist/inhibitor in accordance with the present invention is also meant to encompass any precursor and individual components of the antagonists/inhibitor. For example, if the MST1 antagonist referred to is a peptide, polypeptide or protein such as an antibody, mutant PDX1 or MST1 protein or peptide inhibitor the respective term also includes the polynucleotide encoding such antagonist, the vector, in particular expression vector comprising the coding sequence of the antagonist as well as the host cell comprising the polynucleotide or vector. For example, reference to the use of a PDX1 mutant as an antagonist in accordance with the present invention also includes the use of cells capable of expressing the mutant PDX1 protein. In this case, the MST1 antagonist in accordance with the present invention, i.e. PDX1 mutant and cells expressing the same, respectively, may be used in somatic or, in particular, stem cell therapy of impaired pancreatic function and diabetes or obesity.

Similarly reference to antisense or siRNA as MST1 antagonist in accordance with the present invention includes corresponding vectors such as plasmids encoding and producing the same; see also the Examples. Thus, the term antagonist and inhibitor have to be construed in their broadest sense in that they include any means and methods which the person skilled in the art would consider to bring about the effect of the recited MST1 antagonist.

In embodiment of the present invention, the antagonist is an siRNA comprising or consisting of the nucleotide sequence of any one of SEQ ID NOs 1 to 4: UAAAGAGACCGGCCAGAUU SEQ ID NO: 1, GAUGGGCACUGUCCGAGUA SEQ ID NO: 2, GCCCUCAUGUAGUCAAAUA SEQ ID NO: 3, CCAGAGCUAUGGUCAGAUA SEQ ID NO: 4. Means and methods for the generation of anti-MST1 antibodies and antisense RNA are also described in international application WO 99/15635; see also supra. A plasmid-based method of RNAi encoding shRNAs targeting MST1 for producing MST1 knockdown and regulation of neuronal cell death by MST1-FOXO1 signaling is also described in, e.g., Yuan et al., J. Biol. Chem. 284 (2009), 11285-11292, which also describes the use anti-MST1 monoclonal antibodies. MST1 kinase inhibitors which prevent autophosphorylation of intracellular MST1 are described in international application WO 2012/121992. A dominant negative dnMST1 mutant, i.e. kinase-dead MST1 (K59R; dnMST1) is described in the Examples and in US patent application 2004/0213794 A1.

An MST1 antagonist for use in accordance with the present invention may be validated in vitro and in vivo for their efficacy to restore β-cell survival and/or to reverse diabetes utilizing rodent and human islets and β-cells lines and animal models of T1D and T2D as illustrated in Example 8, using the MST1 antagonists and the pancreas specific MST1 knock-out mouse model exemplified in the Example 1 as controls.

As demonstrated in the Examples, it was found in accordance with the present invention that MST1 directly phosphorylated the β-cell transcription factor PDX1 at amino acid position Thr11, resulting in the ubiquitination and degradation of PDX1, and in a subsequent reduction in PDX1 target genes and loss of glucose-stimulated insulin secretion.

Accordingly, rather than preventing auto-phosphorylation or activation of MST1 it may be beneficial to only interfere with MST1 interaction with PDX1 and MST1 kinase activity in respect to its phosphorylation of PDX1, respectively, thereby possibly remaining its kinase activity towards other substrates and/or other activities for example in functioning as a tumor suppressor unaffected in kind Therefore, in a particular preferred embodiment the MST1 antagonist for use in accordance with the present invention is capable of reducing or inhibiting the binding of MST1 to PDX1 and/or phosphorylation of PDX1 by MST1 at amino acid site Thr11.

As demonstrated in the Examples it was shown that the stabilization of PDX1 leads to a restored β-cell mass and β-cell function. Therefore, it may be beneficial to stabilize PDX1 at amino acid site Thr11 to prevent its degradation and ubiquitination, respectively, thereby supporting PDX1 target gene expression, glucose-stimulated insulin secretion and restoring β-cell mass. Therefore, in another embodiment an antagonist for use in accordance with the present invention is capable of preventing phosphorylation of PDX1 at amino acid site Thr11, which preferably results in stabilization of PDX1 within β-cells compared to PDX1 which is not subject to the MST1 antagonist in accordance with the present invention.

The transcription factor PDX1 (previously called IPF1, IDX1, STF1, or IUF1; see, e.g., Jonsson et al., Nature 371 (1994), 606-609; Stoffers et al., Nat. Genet. 15 (1997), 106-110) is a key factor in β-cell development and function (Johnson et al., J. Clin. Invest. 111 (2003), 1147-1160). The nucleotide and amino acid sequences of PDX1 can be retrieved from publicly available databases, for example the human PDX nucleic acid (and the encoded protein sequences) available as GenBank Accession Nos. U35632 and AAA88820, respectively. Other sources include rat PDX nucleic acid and protein sequences are shown in GenBank Accession No. U35632 and AAA18355, respectively. An additional source includes zebrafish PDX nucleic acid and protein sequences are shown in GenBank Accession No. AF036325 and AAC41260, respectively. The nucleotide and amino acid sequences of PDX1 and its structural characterization are also described in international application WO2000/072885, the disclosure content of which is incorporated herein by reference, in particular with respect to the nucleotide and amino acid sequences of human PDX1. In humans, mutations in PDX1 gene can predispose individuals to develop e.g. maturity onset diabetes of the young (MODY4) (Stoffers et al., Nat. Genet. 17 (1997), 138-139), suggesting a critical role for PDX1 in mature β-cells. Reduced PDX1 expression levels affect insulin expression and secretion and predispose to β-cell apoptosis (Johnson et al., J. Clin. Invest. 111 (2003), 1147-1160; Brissova et al., J. Biol. Chem. 277 (2002), 11225-11232).

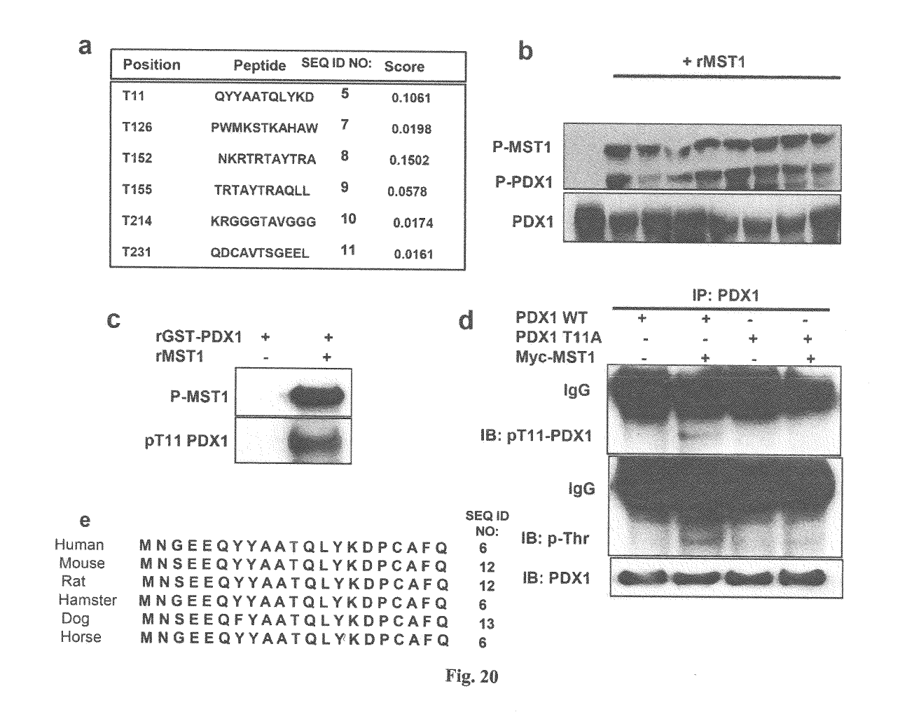

As shown in the Examples MST1-induced PDX1-phosphorylation at the T11 site was markedly reduced in a PDX1-T11A mutant protein. In addition, it could be shown that the mutated PDX1-T11A could reverse the deleterious effects of MST1. Indeed, PDX1-T11A mutant overexpression normalized MST1-induced impairment in GSIS in human islets and INS-1E cells and restored MST1-induced down regulation of PDX1 target genes. Accordingly, in order to have the otherwise beneficial activities of MST1 such as its function as a putative tumor suppressor remain in a preferred embodiment the MST1 antagonist for use in accordance with present invention is a mutant PDX1 wherein the phosphorylation site Thr11 is inactivated (PDX1 T11). Preferably, the mutant PDX1 has the amino acid Thr11 substituted to alanine (PDX1 T11A mutant). In another embodiment, the antagonist of the present invention may be a peptide or peptide mimetic, for example of 10 to 50 amino acids in length comprising the mentioned PDX1 T11 phosphorylation site either functional or inactivated. In one embodiment, the peptide may comprise or substantially consists of the sequence shown in FIG. 20 (a) (QYYAATQLYKD SEQ ID NO: 5) or FIG. 20 (e) (MNGEEQYYAATQLYKDPCAFQ SEQ ID NO: 6). Strategies to design such peptide inhibitors, which copy ‘natural’ motifs that specifically influence kinase activity and/or its intracellular interactions with cognate partners, here of MST1 with PDX1, as an approach for selective inhibition of protein kinases are known in the art and reviewed in, e.g., Finkelman and Eisenstein, Curr. Pharm. Des. 15 (2009), 2463-2470. As used herein, the term “peptide” shall also refer to salts, deprotected form, acetylated form of the peptide, deacetylated form of the peptide, D optical isomer mimetic of the peptide, fusion peptides and hydrates of the above-mentioned peptide. Suitable protecting groups for amino groups are the benzyloxycarbonyl, t-butyloxycarbonyl (BOC), formyl, and acetyl or acyl group. Suitable protecting groups for the carboxylic acid group are esters such as benzyl esters or t-butyl esters. Preferably, the peptide of the present invention does not substantially consists of or comprise 10 to 100 amino acids of the PDX1 amino acid sequence including the Thr11 phosphorylation site or mutant site thereof, more preferably no more than 50, still more preferably no more than 25 amino acids of the PDX1 amino acid sequence including the Thr11 phosphorylation site or mutant site thereof, Typically, the peptide of the present invention substantially consists of or comprise at least 10, more preferably at least 12, still more preferably at least 15 or 20 amino acids of the PDX1 amino acid sequence including the Thr11 phosphorylation site or mutant site thereof

The mutant PDX1 of the present invention can also be used for inducing pancreatic hormone production. For example, international application WO 00/72885, the disclosure content of which is incorporated herein by reference teaches using wild type PDX1 for use in treating a pancreatic associated disorder in a subject in need of pancreatic hormone production in a cell other than an endocrine cell, wherein said cell is selected from a muscle, spleen, kidney, blood, skin or liver cell. Likewise, the mutant PDX1 of the present invention can be used having the advantage of being inert against MST1 mediated degradation. Thus, also in case of gene and stem cell therapy such as taught in WO 00/72885, respectively, comprising genetically engineered expression of PDX1 the mutant PDX1 may be preferred over using the wild type gene and corresponding engineered cells. For example, it has been shown that Pdx1-transfected adipose tissue-derived stem cells (ASCs) differentiate into insulin-producing cells in vivo and reduce hyperglycemia in diabetic mice; see Kajiyama et al., Int. J. Dev. Biol. 54 (2010), 699-705. In particular, STZ-treated mice transplanted with Pdx1-transduced ASCs (Pdx1-ASCs) showed significantly decreased blood glucose levels and increased survival, when compared with control mice. Thus, similarly the PDX1 mutant of the present invention can be used for transfecting ASCs and provide insulin-producing cells for transplantation.

Thus, in a further aspect the present invention relates to the PDX1 T11 mutant described above, to a polynucleotide encoding the PDX1 T11 mutant, to a vector comprising said polynucleotide and to a host cell comprising the polynucleotide or vector. PDX1 recombinant expression vectors and host cells that can be used in accordance with the present invention are disclosed in WO 00/072885, the disclosure content of which is incorporated herein by reference.

Since MST1 seems to ubiquitously expressed including pancreatic β-cells it is prudent to use an MST1 antagonist in accordance with the present invention also in the treatment of subjects who suffer from a metabolic disease, in particular diabetes and/or obesity, but who do not show an enhanced level of MST1 protein and activity, respectively, per se since as shown in the Examples using the mouse models antagonizing the basal level of MST1 activity has a beneficial effect on reducing PDX1 phosphorylation and degradation and thus maintenance of β-cells and improvement of insulin secretion thereby preventing the subject from deleterious effects of the actual cause of the metabolic disease such diabetic condition.

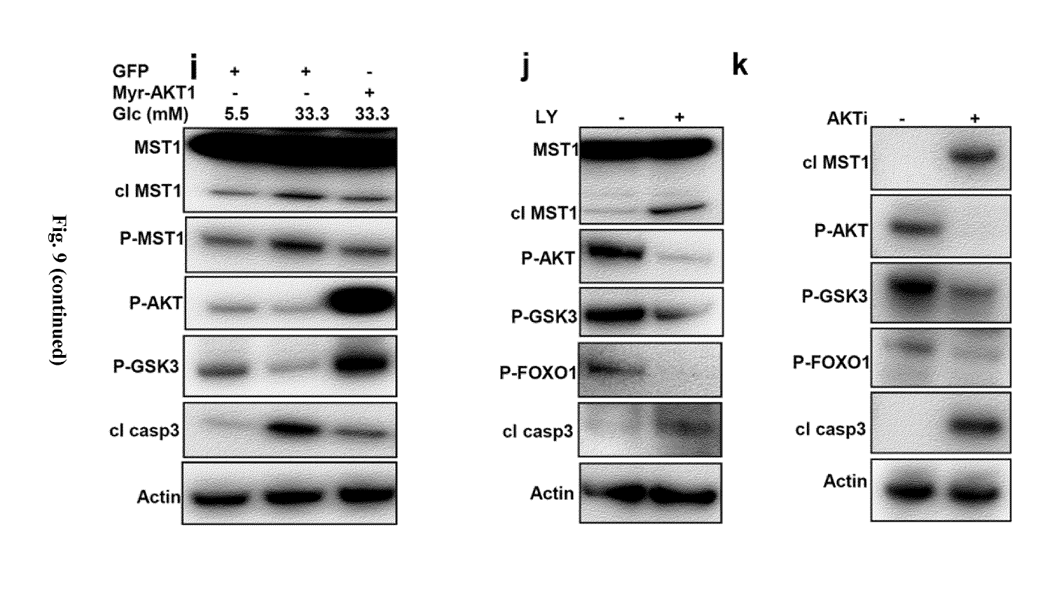

On the other hand, since as also confirmed in the Example 6 and FIG. 10 by overexpression of MST1 an increased level of MST1 activity could itself be the reason for the disease the use an MST1 in accordance with the present invention can be expected to particularly beneficial in the treatment of for example diabetes in a subject having increased MST1 activity. Thus, in one embodiment of the present invention the MST1 antagonist is for use in a subject with increased MST1 activity.

As mentioned above, MST1 and its increased activity may not be the causative event for development of the metabolic disease. In addition, or alternatively the disease might have already progressed such that concomitant treatment with a further therapeutic agent is indicated. For example, symptoms of diabetes mellitus include diabetic ketoacidosis, nonketotic hyperosmolar coma, increased thirst and urination, hunger, weight loss, chronic infections, slow wound healing, fatigue and blurred vision. Furthermore, diabetes is associated with microvascular complications, increased risk of macrovascular complications (ischaemic heart disease, stroke and peripheral vascular disease), and can lead to debilitating and life-threatening complications, e.g. retinopathy leading to blindness, memory loss, chronic renal failure, cardiovascular disease, neuropathy, autonomy dysfunction and limb amputation. In such cases, it will be of benefit to the patient to administer an MST1 antagonist in conjunction with (co-administer) one or more further therapeutic agents which are directed to specific phenotypes of the disease, target the cause of the severity of the disease, for example another target associated with the disease and/or ameliorate the symptoms such as pain the patient is suffering from. For example, in international application WO 2003/059372 the treatment of diabetic late complications, i.e. diabetic neuropathy by the combined treatment with a GLP-1 compound and an aldose reductase inhibitor is described. Likewise, the MST1 antagonist in accordance with the present invention may be used in combination with either or both agents in order to concomitantly improve β-cell mass and insulin secretion.

Therefore, in a further embodiment the present invention relates to a composition comprising an MST1 antagonist or an MST1 antagonist and at least one further therapeutic agent useful in the treatment of a metabolic disease and/or symptoms associate therewith. Preferably, the at least one further therapeutic agent is an anti-diabetic and/or anti-obesity related disease agent. Naturally, the compositions of the present invention are particularly useful in treating and/or preventing metabolic diseases, especially diabetic conditions and obesity or obesity-related diseases, in particular if they are associated with diabetes. In this embodiment, the two or more compounds of the composition may be administer in a single formulation and/or co-administered in separate charges.

Examples of anti-diabetic agents include but are not limited to insulin, insulin sensitizers such as biguanides (metformin) and thiazolidinediones (Rosiglitazone, Pioglitazone), secretagogues such as sulfonylureas, e.g. chlorpropamide (Diabinese), glibenclamide (Glyburide), glimepiride (Amaryl), glipizide (Glucotrol), tolazamide (Tolinase), tolbutamide (Orinase) and glinides, e.g. nateglinide (Starlix) and repaglinide (Prandin). α-glucosidase inhibitors, glucagon-like peptide type (GLP)-1 such as exenatide (Byetta, Bydureon) and liraglutide (Victoza), and incretin-based therapies, e.g. pramlintide (Symlin), dipeptidyl peptidase (DPP) 4 inhibitors and bromocriptine. Generally anti-diabetic medications treat diabetes mellitus by lowering blood glucose and the levels of other known risk factors that damage blood vessels. With the exceptions of insulin, exenatide, liraglutide and pramlintide, all are administered orally (oral hypoglycemic agents or oral anti-hyperglycemic agents).

Examples of anti-obesity and/or anti-obesity related disease agents include but are not limited to natural products, natural product mimetics, synthetic small molecules, and peptides/hormones reviewed in Gonzalez-Castejón et al., Pharmacol. Res. 64 (2011) 438-55 and Oh et al., Curr. Top. Med. Chem. 9 (2009), 466-81, the disclosure content of which is incorporated herein by reference.

Selecting an appropriate anti-diabetic drug depends on the nature of the diabetes, age and the individual's overall health status. A combination therapy is particularly preferred during the progress of diabetes. For example, T1D is caused by the lack of insulin. Therefore, insulin must be substituted by subcutaneous injections. In contrast, T2D is a disease of insulin resistance by cells. In this case, treatment options include (1) agents that increase the amount of insulin secreted by the pancreas, (2) agents that increase the sensitivity of target organs to insulin, and (3) agents that decrease the rate at which glucose is absorbed from the gastrointestinal tract. The therapeutic combination in T2D may include insulin, not necessarily because oral agents have failed completely, but in search of a desired combination of effects.

Therefore, the advantages of the present invention comprising the administration of an MST1 antagonist and or an MST1 antagonist and an anti-diabetic agent or an anti-obesity and/or anti-obesity related disease agent, i.e. combination therapy are that (1) better glycemic control can be obtained with a combination of two drugs that work at different sites, (2) there are fewer side effects with lower doses of two drugs than there would be from a large dose of one drug (synergistic effect), and (3) if these drugs are combined in the same pill or capsule there will not only be better compliance, but also the cost will be lower (Bell et al., Diabetes Rev. 7 (1999), 94-113). In the context of the present application, “co-administration” of two or more compounds is defined as administration of the two or more compounds to the patient within 24 h, including separate administration of two medicaments each containing one of the compounds as well as simultaneous administration whether or not the two compounds are combined in one formulation or whether they are in two separate formulations. A “synergistic effect” of two compounds is in terms of statistical analysis an effect which is greater than the additive effect which results from the sum of the effects of the two individual compounds.

As mentioned above, the MST1 antagonist and composition of the present invention can be used in in the treatment of a variety of metabolic diseases with an emphasis on diabetic conditions and obesity as well as symptoms associated therewith. Thus, the medical indications include but are not limited to
preventing, slowing the progression of, delaying or treating a metabolic disorder or disease, such as e.g. type 1 diabetes (T1D), type 2 diabetes (T2D), maturity onset diabetes of the young (MODY), latent autoimmune diabetes with onset in adults (LADA), insulin dependent diabetes mellitus (IDDM), non-insulin dependent diabetes mellitus (NIDDM) or Gestational diabetes mellitus (GDM), impaired glucose tolerance (IGT), impaired fasting blood glucose (IFG), hyperinsulinemia, insulin resistance, hyperglycemia, postprandial hyperglycemia, postabsorptive hyperglycemia, overweight, obesity, dyslipidemia, hyperlipidemia, hypercholesterolemia, hypertriglyceridemia, hypertension, endothelial dysfunction, metabolic syndrome, new onset diabetes after transplantation (NODAT) and complications associated therewith, and post-transplant metabolic syndrome (PTMS) and complications associated therewith;
improving and/or maintaining glycemic control and/or for reducing of fasting plasma glucose, of postprandial plasma glucose, of postabsorptive plasma glucose and/or of glycosylated hemoglobin HbA 1 c;
preventing, slowing, delaying or reversing progression from pre-diabetes, impaired glucose tolerance (IGT), impaired fasting blood glucose (IFG), insulin resistance and/or from metabolic syndrome to type 2 diabetes mellitus;
preventing, reducing the risk of, slowing the progression of, delaying or treating of complications of diabetes mellitus such as micro- and macrovascular diseases, such as nephropathy, micro- or macroalbuminuria, proteinuria, retinopathy, cataracts, neuropathy;
preventing, slowing, delaying or treating the degeneration of pancreatic beta cells and/or the decline of the functionality of pancreatic beta cells and/or for improving, preserving and/or restoring the functionality of pancreatic beta cells and/or stimulating and/or restoring or protecting the functionality of pancreatic insulin secretion;
preventing, slowing the progression of, delaying or treating type 2 diabetes with failure to conventional anti-diabetic mono- or combination therapy;
achieving a reduction in the dose of conventional anti-diabetic medication required for adequate therapeutic effect;
reducing the risk for adverse effects associated with conventional anti-diabetic medication (e.g. hypoglycemia or weight gain); and/or
maintaining and/or improving the insulin sensitivity and/or for treating or preventing hyperinsulinemia and/or insulin resistance.

Moreover, it is expected that MST1 antagonists alone or in combination with aforementioned anti-diabetic agents and/or anti-obesity agents according to the invention can be used to treat infertility and to improve fertility, respectively, in humans or mammals, particularly if the infertility is connected with insulin resistance or with polycystic ovary syndrome. On the other hand these substances are suitable for influencing sperm motility and are thus suitable for use as male contraceptives. In addition, the substances are suitable for treating growth hormone deficiencies connected with restricted growth, and may reasonably be used for all indications for which growth hormone may be used.

The dosage regimen utilizing the MST1 antagonist in accordance with the present invention is selected in accordance with a variety of factors including type, species, age, weight, sex and medical condition of the patient; the severity of the condition to be treated; the route of administration; and the particular compound employed. It will be acknowledged that an ordinary skilled physician can easily determine and prescribe the effective amount of the compound required to prevent, counter or arrest the progress of the condition. The term “subject” and “patient” is used interchangeably herein and means an individual in need of a treatment of a metabolic disease. Preferably, the subject is a mammal, particularly preferred a human.

“Treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. As used herein, the terms “treat” or “treatment” refer to both therapeutic treatment and prophylactic or preventative measures, wherein the object is to prevent or slow down (lessen) an undesired physiological change or disorder, such as the development or spread of a metabolic disease. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the manifestation of the condition or disorder is to be prevented.

For use as a pharmaceutical composition, the MST1 antagonist according to the invention, optionally combined with other active agents, may be incorporated together with one or more inert conventional carriers and/or diluents. Pharmaceutically acceptable carriers and administration routes can be taken from corresponding literature known to the person skilled in the art. The pharmaceutical compositions of the present invention can be formulated according to methods well known in the art; see for example Remington: The Science and Practice of Pharmacy (2000) by the University of Sciences in Philadelphia, ISBN 0-683-306472, Vaccine Protocols, 2nd Edition by Robinson et al., Humana Press, Totowa, N.J. USA, 2003; Banga, Therapeutic Peptides and Proteins: Formulation, Processing, and Delivery Systems. 2nd Edition by Taylor and Francis. (2006), ISBN: 0-8493-1630-8. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected by different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods. Pharmaceutical compositions for oral administration, such as single domain antibody molecules (e.g., “Nanobodies™”) etc. are also envisaged in the present invention. Such oral formulations may be in tablet, capsule, powder, liquid or semi-solid form. A tablet may comprise a solid carrier, such as gelatin or an adjuvant. Further guidance regarding formulations that are suitable for various types of administration can be found in Remington's Pharmaceutical Sciences, Mace Publishing Company, Philadelphia, Pa., 17th ed. (1985) and corresponding updates. For a brief review of methods for drug delivery see Langer, Science 249 (1990), 1527-1533. In one embodiment, the MST1 antagonist and composition of the invention is administered to a human patient once daily, each other day, thrice weekly, twice weekly or once weekly, preferably less than once daily.

In a further embodiment, the present invention relates to a dietary food product including beverages comprising an MST1 antagonist or a composition of the present invention. Conventional diet foods involve products containing ingredients which give a feeling of fullness and yet have low caloric values, products containing low caloric sweeteners as a substitute for sugar, and products containing drugs having anorexic or sweetness-repellent effects. Many of conventional diabetic foods for regulating total calorie intake are unappetizing. Although calorie intake can be easily controlled in the hospital, preparation of calorie-restricted foods, injection of insulin for inhibiting an increase in blood glucose level and intake of drugs impose serious burden and stress both in mind and body of patients after discharge from the hospital.

In order to overcome the mentioned disadvantages, food products may be supplement with an MST1 antagonist or a composition of the present invention, thereby for example substituting ingredients which influence glucose uptake or consumption. In this context, common basic dietary food components may be used in accordance with the present invention such as dietary fibers; see, e.g., European patent application EP 1 167 536 A and international application WO 2011/109900. Thus, the dietary food of the present invention may be a medical food product or functional food such as is used by athletes or also common people for reducing body weight and/or enhancing the coenaesthesis. However, it is also envisaged to supplement normal calorie rich food with MST1 antagonist or a composition of the present invention in order to improve consumption of the food and energy after food take up and keep the subject well.

Thus, in a further embodiment of the present invention the MST1 antagonist, composition and dietary food product disclosed herein are used for reducing body weight and/or enhancing the coenaesthesis.

In one embodiment, the MST1 antagonist in accordance with the present invention is administered to a human patient once daily, each other day, thrice weekly, twice weekly or once weekly, preferably less than once daily.

In addition, or alternatively the dosage of the MST1 antagonist for therapeutic use or in the composition and dietary food product is present in an amount of about 0.05 mg per kilogram body weight per day to 500.0 mg per kilogram body weight per day. In one embodiment the dosage of the MST1 antagonist is in an amount of about 0.05 mg per kilogram body weight per day to 25 mg per kilogram body weight per day. In preferred embodiment the dosage of the MST1 antagonist is less than 1 mg per kilogram by weight per day.

Further embodiments of the present invention will be apparent from the definitions and Examples that follow.

### EXAMPLES

The practice of the present invention will employ, unless otherwise indicated, conventional techniques of cell biology, cell culture, molecular biology, transgenic biology, microbiology, recombinant DNA, and immunology, which are within the skill of the art.

For further elaboration of general techniques useful in the practice of this invention, the practitioner can refer to standard textbooks and reviews in cell biology and tissue culture; see also the references cited in the Examples. General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Non-viral Vectors for Gene Therapy (Wagner et al. eds., Academic Press 1999); Viral Vectors (Kaplitt & Loewy eds., Academic Press 1995); Immunology Methods Manual (Lefkovits ed., Academic Press 1997); and Cell and Tissue Culture: Laboratory Procedures in Biotechnology (Doyle & Griffiths, John Wiley & Sons 1998). Reagents, cloning vectors and kits for genetic manipulation referred to in this disclosure are available from commercial vendors such as BioRad, Stratagene, Invitrogen, Sigma-Aldrich, and ClonTech.

### Supplementary Materials and Methods

### Cell Culture, Treatment and Islet Isolation

Human islets were isolated from twenty pancreases of healthy organ donors and from five with T2D at the University of Illinois at Chicago or Lille University and cultured on extracellular matrix (ECM) coated dishes (Novamed, Jerusalem, Israel) as described previously (Schulthess et al., Cell. Metab. 9 (2009) 125-139). Islet purity was greater than 95% as judged by dithizone staining (if this degree of purity was not achieved by routine isolation, islets were handpicked). Islets from MST1^{-/-} mice and their WT littermates were isolated as described previously (Schulthess et al., Cell. Metab. 9 (2009) 125-139). Pancreata were perifused with a Liberase™ (#05401119001, Roche, Mannheim, Germany) solution according to the manufacturer's instructions and digested at 37° C., followed by washing and handpicking Human islets were cultured in complete CMRL-1066 (Invitrogen) medium at 5.5 mM glucose and mouse islets and INS-1E cells at complete RPMI-1640 medium at 11.1 mM glucose and HEK293 cells were cultured in Dulbecco's modified Eagle's medium (DMEM). All media included with glutamate, 1% penicillin-streptomycin and 10% fetal bovine serum (FBS, all PAA). INS-1E medium was supplemented with 10 mM HEPES, 1 mM sodium pyruvate and 50 μM β-mercaptoethanol. Islets and INS-1E were exposed to complex diabetogenic conditions: 22.2-33.3 mM glucose, 0.5 mM palmitic acid, the mixture of 2 ng/ml recombinant human IL-1β (R&D Systems, Minneapolis, Minn.)+1,000 U/ml recombinant human IFN-γ (PeProTech) for 72 h, 100 μM H₂O₂ for 6 h, 1 mM streptozotocin (STZ) for 8 h or 1 mM thapsigargin for 6 h (all Sigma). In some experiments, cells were additionally cultured with 10-25 μM JNK selective inhibitor SP600125, 25 μM selective PI-3 kinase inhibitor LY294002, 20 μM AKT inhibitor V, Triciribine, selective AKT1/2/3 inhibitor, 25 μM pan-caspase inhibitor Z-VAD (OMe)-fmk, 100 μM Bax-inhibiting peptide V5 or Bax-inhibiting peptide, negative control, InSolution™ MG-132, proteasome inhibitor (all Calbiochem), 100 nM Glucagon like-peptide 1 (GLP1), 100 nM recombinant human insulin and cycloheximide (CHX) (all Sigma). Palmitic acid was dissolved as described previously (Maedler et al., Diabetes 50 (2001) 69-76). Ethical approval for the use of islets had been granted by the Ethics Committee of the University of Bremen.

### Animals

For MLD-STZ experiment, 8-10 week old MST1^{-/-} mice on a 129/sv genetic background (Dong et al., 183 (2009) 3865-72) and their MST1^{+/+} WT littermates were i.p. injected with streptozotocin (STZ; 40 mg/kg; Sigma) freshly dissolved in 50 mM sodium citrate buffer (pH 4.5) or citrate buffer as control for 5 consecutive days (referred to as multiple low dose/MLD-STZ). For the high fat diet (HFD) experiments, 8-10 week old MST1^{-/-} mice and their MST1^{+/+} WT littermates were fed a normal diet (ND, Harlan Teklad Rodent Diet 8604, containing 12.2, 57.6 and 30.2% calories from fat, carbohydrate and protein, respectively) or a high fat/high sucrose diet (HFD, “Surwit” Research Diets, New Brunswick, N.J., containing 58, 26 and 16% calories from fat, carbohydrate and protein). After 16 weeks of HFD feeding, a single dose of 100 mg/kg body weight STZ was i.p. injected to induce β-cell failure and insulin deficiency. Three weeks after STZ injection, wild-type HFD/STZ-treated mice displayed hyperglycemia, insulin resistance and glucose intolerance. For both models, random blood was obtained from the tail vein of non-fasted mice and glucose was measured using a Glucometer (Freestyle; TheraSense Inc., Alameda, Calif.). Mice were killed at the end of experiment, pancreas was isolated. Throughout the whole study, food consumption and body weight were measured weekly. To create β-cell-specific MST1^{-/-} mice, mice harboring exon 4 of the MST1 gene flanked by loxP sites (MST19^{fl/fl}) (Dong et al., 183 (2009) 3865-72) were crossed with mice expressing cre under the rat insulin-2 promoter (B6;D2-Tg(Ins-cre)23Herr: RIP-Cre (Herrera et al., Development 127 (2000) 2317-2322). RIP-Cre-MST1^{fl/-} mice were intercrossed to generate RIP-Cre-MST1^{fl/fl}. Mice were MLD-STZ injected as described above. All animals were housed in a temperature-controlled room with a 12 h light/dark cycle and were allowed free access to food and water in agreement to NIH animal care guidelines of the §8 German animal protection law and approved by the Bremen Senate.

### Intraperitoneal Glucose and Insulin Tolerance Tests and Measurement of Insulin Release

For intraperitoneal glucose tolerance tests (ipGTTs), mice were fasted 12 h overnight and injected i.p. with glucose (40%; B. Braun, Melsungen, Germany) at a dose of 1 g/kg body weight. Blood samples were obtained at time points 0, 15, 30, 60, 90, and 120 min for glucose measurements using a Glucometer and at time points 0 and 30 min for measurement of serum insulin levels. For i.p. insulin tolerance tests, mice were injected with 0.75 U/kg body weight recombinant human insulin (Novolin, Novo Nordisk) after 5 h fasting, and glucose concentration was determined with the Glucometer. Insulin secretion was measured before (0 min) and after (30 min) i.p. injection of glucose (2 g/kg) and measured using ultrasensitive mouse Elisa kit (ALPCO Diagnostics, Salem, N.H.).

### Plasmids

pCMV-myc-MST1 and kinase-dead (MST1-K59; dnMST1) are described in Yamamoto et al., J. Clin. Invest. 111 (2003), 1463-1474. Mouse pB.RSV.PDX1-GFP is described in Kawamori et al., Diabetes 52 (2003) 2896-2904. pcDNA3 Myr-HA Akt1, HA-Ubiquitin and pCDNA3 Jnk1a1 (apf)(dn-JNK) plasmids were obtained from Addgene (Cambridge, Mass.). Mouse PDX1 mutants (T11, T126, T152, T155, T214 and T231) in pCGIG5 vector were generated by site-directed mutagenesis as described previously (Frogne et al., 7 (2012), e35233). All mutations were verified by sequencing. To make bacterial expression plasmids for PDX1 mutants, the complete mouse PDX1 CDS (wild type and mutants) has been amplified by PCR using a specific set of primers from pCGIG5 plasmids and cloned into a pGEX-6P-1 bacterial expression vector. The rat insulin driven luciferase vector (RIP-Luc) was constructed by subcloning a 700 by fragment containing −660 bp of the rat 2 insulin promoter into a pMCS-Green-Renilla-Luc vector (Thermo Scientific). pCMV-Red firefly Luc vector was obtained from Thermo Scientific.

### Transfections

To knockdown MST1 in human islets, SMARTpool technology from Dharmacon was used. A mix of ON-TARGETplus siRNAs directed against the following sequences in human MST1: UAAAGAGACCGGCCAGAUU SEQ ID NO: 1, GAUGGGCACUGUCCGAGUA SEQ ID NO: 2, GCCCUCAUGUAGUCAAAUA SEQ ID NO: 3, CCAGAGCUAUGGUCAGAUA SEQ ID NO: 4 (100 nM, Dharmacon) was transiently transfected into human islets and efficiently reduced MST1 levels. An ON-TARGETplus non-targeting siRNA pool from Dharmacon served as a control. To knock down Bim and caspase-3 in human islets, siRNA targeting human Bim (SignalSilence Bim SiRNA I, Cell Signaling) and caspase-3 (NEB) was used. GFP, MST1, do-MST1 (K59), dn-JNK1 and Myr-Akt1 plasmids were used to overexpress these proteins in human islets and INS1E cells. An adapted improved protocol to achieve silencing and overexpression in human islets was developed (Shu et al., Diabetes 57 (2007), 645-53). Islets were partially dispersed with accutase (PAA) to break islets into smaller cell aggregates to increase transfection efficiency and cultured on ECM dishes for at least 2 days. Isolated islets and INS1E cells were exposed to transfection Ca²⁺-KRH medium (KCl 4.74 mM, KH₂PO₄ 1.19 mM, MgCl₂6H₂O 1.19 mM, NaCl 119 mM, CaCl₂ 2.54 mM, NaHCO₃ 25 mM, HEPES 10 mM). After 1 h incubation, lipoplexes (Lipofectamine2000, Invitrogen)/-siRNA ratio 1:20 pmol or -DNA ratio 2.5:1) were added to transfect the islets and INS1 cells. After additional 6 h incubation, CMRL-1066 or RPMI-1640 medium containing 20% FCS and L-Glutamine were added to the transfected islets or INS1 cells. Efficient transfection was evaluated based on Fluorescein-labeled siRNA (NEB) or eGFP positive cells analyzed by fluorescent or confocal microscopy. HEK293 were transiently transfected using Optimem medium and Lipofectamine (Invitrogen) according to the manufacturer's instructions.

### Glucose Stimulated Insulin Secretion

For acute insulin release in response to glucose, primary human and mouse islets and INS1 cells were washed and pre-incubated (30 min) in Krebs-Ringer bicarbonate buffer (KRB) containing 2.8 mM glucose and 0.5% BSA. KRB was then replaced by KRB 2.8 mM glucose for 1 h (basal), followed by an additional 1 h in KRB 16.7 mM glucose. Insulin content was extracted with 0.18N HCl in 70% ethanol. Insulin was determined using human and mouse insulin ELISA (ALPCO Diagnostics, Salem, N.H.). Secreted insulin was normalized to insulin content.

### Immunohistochemistry

Pancreatic tissues were processed as previously described (Shu et al., Diabetes 57 (2007), 645-53). In brief, mouse pancreases were dissected and fixed in 4% formaldehyde at 4° C. for 12 h before embedding in paraffin. Human and mouse 4-μm sections were deparaffinized, rehydrated and incubated overnight at 4° C. with anti-insulin (Dako), anti-P-MST1 (Cell Signaling), anti-Bim (Cell Signaling), anti-PDX-1 (abcam), anti-glucagon (Dako), anti-glut2 (Chemicon) and anti-mouse anti-Ki67 (BD Pharmingen) antibodies followed by fluorescein isothiocyanate (FITC)- or Cy3-conjugated secondary antibodies (Jackson ImmunoResearch Laboratories, West Grove, Pa.). Slides were mounted with Vectashield with 4′6-diamidino-2-phenylindole (DAPI) (Vector Labs). β-cell apoptosis for mouse sections or primary islets cultured on ECM dishes was analyzed by the terminal deoxynucleotidyl transferase-mediated dUTP nick-end labeling (TUNEL) technique according to the manufacturer's instructions (In Situ Cell Death Detection Kit, TMR red; Roche) and double stained for insulin. Fluorescence was analyzed using a Nikon MEA53200 (Nikon GmbH, Dusseldorf, Germany) microscope and images were acquired using NIS-Elements software (Nikon).

### Morphometric Analysis

For morphometric data, ten sections (spanning the width of the pancreas) per mouse were analyzed. Pancreatic tissue area and insulin-positive area were determined by computer-assisted measurements using a Nikon MEA53200 (Nikon GmbH, Dusseldorf, Germany) microscope and images were acquired using NIS-Elements software (Nikon). The number of islets (defined as insulin-positive aggregates at least 25 μm in diameter) was scored and used to calculate islet density (number of islets per square centimeter of tissue), mean islet size (the ratio of the total insulin-positive area to the total islet number on the sections). Mean percent β-cell fraction per pancreas was calculated as the ratio of insulin-positive and whole pancreatic tissue area. β-cell mass was obtained by multiplying the β-cell fraction by the weight of the pancreas. Morphometric β-cell and islet characterizations are results from analyses of at least 100 islets per mouse.

### Western Blot Analysis

At the end of the incubation periods, islets and INS1E cells were washed in ice-cold PBS and lysed in lysis buffer containing 20 mM Tris acetate, 0.27 M sucrose, 1 mM EDTA, 1 mM EGTA, 50 mM NaF, 1% Triton X-100, 5 mM sodium pyrophosphate and 10 mM β-glycerophosphate. Prior to use, the lysis buffer was supplemented with Protease- and Phosphatase-inhibitors (Pierce, Rockford, Ill., USA). Protein concentrations were determined with the BCA protein assay (Pierce). Equivalent amounts of protein from each treatment group were run on a NuPAGE 4-12% Bis-Tris gel (Invitrogen) and electrically transferred onto PVDF membranes. After 1 h blocking at room temperature using 5% milk (Cell Signaling), membranes were incubated overnight at 4° C. with rabbit anti-MST1, rabbit anti-P-MST1, rabbit anti-Bim, rabbit anti-P-AKT (Ser437), rabbit anti-Bax, rabbit anti-Bcl-2, rabbit anti-Bcl-xL, rabbit anti-Bad, rabbit anti-phospho Bad, rabbit anti-PUMA, rabbit anti-Bak, rabbit anti-Mcl1, rabbit anti-pan-phopsho threonine, mouse monoclonal anti-pan-phospho threonine, rabbit anti-phospho GSK-3, rabbit anti-phospho FOXO1, mouse anti-Myc, rabbit anti-cleaved caspase-3, rabbit anti-cleaved caspase-9, rabbit anti-cytochrome c, rabbit anti-cytochrom oxidase (COX), rabbit anti-phospho JNK (Thr183/Tyr185), rabbit anti-phospho c-Jun (Ser63), rabbit anti-PARP, rabbit anti-tubulin, rabbit anti-GAPDH and rabbit anti-β-actin (all Cell Signaling Technology), rabbit anti-P-MST1, rabbit anti-GFP, mouse anti-NOXA and rabbit anti-PDX1 (Abcam), rabbit anti-P-H2B (Millipore) and rabbit anti-P-PDX1 (Thr11) (Abgent) antibodies, followed by horseradish-peroxidase-linked anti-rabbit or mouse IgG (Jackson). Membrane was developed using a chemiluminescence assay system (Pierce) and analyzed using DocIT®LS image acquisition 6.6a (UVP BioImaging Systems, Upland, Calif., USA).

### Immunoprecipitation

For immunoprecipitation, cells were washed with PBS and lysed in cold buffer containing 20 mM Tris-HCl (pH 7.5), 150 mM NaCl, 0.27 M sucrose, 1 mM EDTA, 1 mM EGTA, 50 mM NaF, 1% NP-40, 5 mM sodium pyrophosphate and 10 mM β-glycerophosphate supplemented with proteinase/phosphatase inhibitors for 30 min on ice. Lysates were centrifuged at 12,000 g for 15 min at 4° C. prior to immunoprecipitation. Immunoprecipitations were carried out with incubating 0.5-1 mg of total lysate with rabbit anti-PDX1 (1:500), rabbit anti-MST1 (1:50), mouse anti-Myc (1:1000) and rabbit anti-GFP (1:1000) antibodies on a rotator at 4° C. overnight. Immunocomplexes were then captured with Protein A Agarose Fast Flow (Millipore) by rotation at 4° C. for 4 h. After five washes with cold lysis buffer, the immunoprecipitates were used for kinase assays or resuspended in sample buffer and separated by NuPAGE 4-12% Bis-Tris gels (Invitrogen).

### In Vitro Kinase Assay

Purified human active MST1 (Upstate Biotechnology) was incubated with ³²P-ATP (2 μCi, Perkin Elmer Life Sciences), ATP (100 μM) and 1 mM dithiothreitol (DTT) in a kinase buffer containing 40 mM HEPES (pH 7.4), 20 mM MgCl₂, 1 mM EDTA and 1 μg of purified recombinant human PDX-1 (Abcam) or bacterially purified GST-PDX1 (WT and mutants) as substrates. After incubation at 30° C. for 30 min, the reaction was stopped by adding loading buffer and proteins were separated on NuPAGE gels and phosphorylation levels visualized either by autoradiography or specific antibody for phospho-PDX1. The total PDX1 was detected with anti-PDX1 antibody.

### In Vivo Kinase Assay

HEK293 cells were transiently transfected with PDX1 and MST1 expression plasmids. Then, cell lysates were subjected to immunoprecipitation with anti-PDX1 antibody. The immunoprecipitates were separated by NuPAGE Bis-Tris gels and transferred to PVDF membranes and subsequently subjected to analyses of phosphorylation levels by pan phospho-threonine antibody, which binds to threonine-phosphorylated sites in a manner largely independent of the surrounding amino-acid sequence or pan phospho-serine antibody which is recognizes serine-phosphorylated proteins.

### In Vivo Ubiquitination

HEK293 cells were cultured in 10-cm cell culture dishes and transfected with HA-ubiquitin, PDX1 and MST1 expression plasmids for 48 h. For ubiquitination in human islets, 5000 islets per condition were transfected with ubiquitin plasmid. After 24 h, islets were infected with Ad-GFP or Ad-MST1 for 6 h and kept for another 48 h. HEK293 cells and islets were exposed to 20 μM MG-132 for the last 6 h of the experiment. Lysates were immunoprecipitated with PDX1-specific antibody overnight at 4° C. Immunocomplexes were then captured with Protein A Agarose by rotation at 4° C. for 4 h. After extensive washing, immunoprecipitates were boiled in sample buffer and proteins subjected to western blotting with ubiquitin-specific antibody.

### Protein Degradation Analysis

HEK293 cells were transfected with PDX1 alone, or together with MST1 expressing-plasmids. Human islets were infected with Ad-GFP (control) or Ad-MST1. At 48 h after post-transfection/infection, cells were treated with 50 μg/ml translation initiation inhibitor cycloheximde (CHX) to the medium at the times indicated and the lysates were subjected to western blotting.

### RNA Extraction and RT-PCR Analysis

Total RNA was isolated from cultured human islets and INS1 cells using TRIzol (Invitrogen), and RT-PCR performed as described previously (Shu et al., Diabetologica 55 (2012), 3296-307). For analysis, the Applied Biosystems StepOne Real-Time PCR system (Applied Biosystems, CA, USA) with TaqMan® Fast Universal PCR Master Mix for TaqMan assays (Applied Biosystems) was used. TaqMan® Gene Expression Assays were used for pdx1 (Hs00426216_m1), SLC2A2 (Hs01096905_m1), GCK (Hs01564555_m1), insulin (Hs02741908_m1), PPIA (Hs99999904_m1) and tubulin (Hs00362387_m1) for human and PDX1 (Rn00755591_m1), SLC2A2 (Rn00563565_m1), GCK (Rn00688285_m1), INS1 (Rn02121433_g1), INS2 (Rn01774648_g1), BCL2L11 (Hs01083836_m1), PPIA (Rn00690933_m1) and tuba1a (Rn01532518_g1) for rat.

### Luciferase Reporter Assay

The transcriptional activity of the PDX1 at promoter level was evaluated using rat Ins2-Luc renilla reporter gene HEK293 cells were transfected with Ins2-Luc renilla, pCMV-firefly, PDX1-WT or PDX1-T11A, alone or together with Myc-MST1 expressing plasmids for 48 h. INS-1E cells transfected with Ins2-Luc renilla and pCMV-firefly plasmids and were infected with Ad-GFP or Ad-MST1 for 48 h. Luciferase activity determined using the Renilla-Firefly Luciferase Dual Assay Kit according to the manufacturer's instructions (Pierce). pCMV-firefly was used as transfection control.

### Adenovirus Infection

Isolated human islets and INS1E cells were infected with adenovirus carrying e-GFP as a control or MST1 (AdX-MST1) at a multiplicity of infection (MOI) of 20 (for INS1E) or 100 (for human islets) for 4 h. Adenovirus was subsequently washed off with PBS and replaced by fresh medium with 10% FBS and GSIS or RNA and protein isolation performed after 48 h or 72 h post-infection.

### Purification of GST-PDX1 Recombinant Proteins

Expression and induction of recombinant GST proteins were performed as described previously (Tolia et al., Nat. Methods 3 (2006), 55-64). Escherichia coli BL21 cells with various GST-fusion expression plasmids were cultured at 37° C. and expression of recombinant proteins was induced by 0.1 mM final concentration of Isopropyl-β-D-thio-galactoside (IPTG; sigma) for 2.5 h. Cells were lysed using B-PER bacterial protein extraction reagent (Pierce) and purified using Glutathione Spin Columns (Pierce).

### Cytochrome c Release

Cytochrome c release was performed by digitonin-based subcellular fractionation technique. Briefly, INS1 cells were digitonin-permeabilized for 5 min on ice after resuspension of the cell pellet in 200 μl of cytosolic extraction buffer (CEB: 250 mM sucrose, 70 mM KCl, 137 mM NaCl, 4.3 mM Na₂HPO₄, 1.4 mM KH₂PO₄ (pH 7.2), with 300 μg/ml digitonin (Sigma). Cells were then centrifuged at 1000 g for 5 min at 4° C. Supernatants (cytosolic fractions) were collected and pellets solubilized in the same volume of mitochondrial lysis buffer (MLB: 50 mM Tris, pH 7.4, 150 mM NaCl, 2 mM EDTA, 2 mM EGTA, 0.2% Triton X-100, 0.3% NP-40), followed by centrifugation at 10,000 g for 10 min at 4° C. After centrifugation, supernatants, which are the heavy membrane fractions enriched for mitochondria as well as cytosolic fractions were subjected to western blot analysis.

Generation of Stably Expressed shRNAmir-MST1 INS1 Cell Line

To knock down MST1 expression in INS1E cells, lentiviral shRNAmir targeting MST1 or control shRNAmir vectors (pGIPZ collection, Open Biosystems, Huntsville, Ala.) were transfected into INS-1E cells and stable clones were generated by selection with puromycine (1 to 2.5 μg/ml). Positive clonal cell lines were identified by immunoblotting using antibody directed against MST1. After selection, INS1E lines were maintained in culture medium containing 1.5 μg/ml puromycin.

### Statistical Analysis

Data are presented as means±SE. Mean differences were tested by Student's t-tests. To account for multiplicity in the treated cells in vitro and mice in vivo, a Bonferroni correction used.

### Example 1

### Inhibition of MST1 Activity in β-Cells Prevents Hyperglycemia and Diabetes

To test whether β-cell specific inhibition of MST1 activity prevents hyperglycemia and diabetes progression β-cell specific MST1^{-/-} mice were generated (FIGS. 1 and 2). In brief, β-cell specific MST1^{-/-} mice were created by mice harboring exon 4 of the MST1 gene flanked by loxP sites (MST1^{fl/fl}) and were crossed with mice expressing cre under the rat insulin-2 promoter (B6;D2-Tg(Ins-cre)23Herr: RIP-Cre (Herrera et al., Development 127 (2000), 2317-2322). RIP-Cre-MST1^{fl/-} mice were intercrossed to generate RIP-Cre-MST1^{fl/fl}. For the MLD-STZ (T1D) model, mice were i.p. injected with streptozotocin (STZ; 40 mg/kg; Sigma) freshly dissolved in 50 mM sodium citrate buffer (pH 4.5) or citrate buffer as control for 5 consecutive days (referred to as multiple low dose/MLD-STZ). All animals were housed in a temperature-controlled room with a 12 h light/dark cycle and were allowed free access to food and water in agreement to NIH animal care guidelines of the §8 German animal protection law and approved by the Bremen Senate.

### Example 2

### Inhibition of MST1 Activity Protects from Diabetes

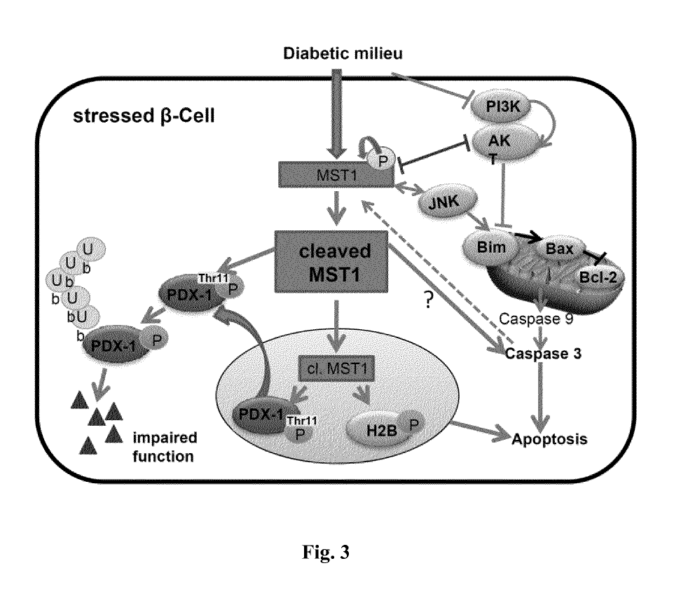

To test whether inhibition of MST1 activity protects from diabetes in vivo, a MLD-STZ experiment as described in Example 1 was performed in 8-10 week old MST1^{-/-} mice on a 129/sv genetic background and their MST1^{+/+} WT littermates (FIGS. 3 and 4). For the high fat diet (HFD) experiments, 8-10 week old MST1^{-/-} mice and their MST1^{+/+} WT littermates were fed a normal diet (ND, Harlan Teklad Rodent Diet 8604, containing 12.2, 57.6 and 30.2% calories from fat, carbohydrate and protein, respectively) or a high fat/high sucrose diet (HFD, “Surwit” Research Diets, New Brunswick, N.J., containing 58, 26 and 16% calories from fat, carbohydrate and protein, respectively). After 16 weeks of HFD feeding, a single dose of 100 mg/kg BW STZ was i.p. injected to induce β-cell failure and insulin deficiency. Three weeks after STZ injection, WT HFD/STZ-treated mice displayed hyperglycemia, insulin resistance and glucose intolerance. For both models, random blood was obtained from the tail vein of non-fasted mice and glucose was measured using a Glucometer (Freestyle; TheraSense Inc., Alameda, Calif.). Mice were killed at the end of experiment, pancreas was isolated. Throughout the whole study, food consumption and body weight were measured weekly. For i.p. ipGTT, mice were fasted 12 h overnight and injected i.p. with glucose (40%; B. Braun, Melsungen, Germany) at a dose of 1 g/kg body weight. Blood samples were obtained at time points 0, 15, 30, 60, 90, and 120 min for glucose measurements using a Glucometer and at time points 0 and 30 min for measurement of serum insulin levels. For i.p. insulin tolerance tests, mice were injected with 0.75 U/kg body weight recombinant human insulin (Novolin, Novo Nordisk) after 5 h fasting, and glucose concentration was determined with the Glucometer. Insulin secretion was measured before (0 min) and after (30 min) i.p. injection of glucose (2 g/kg) and measured using ultrasensitive mouse Elisa kit (ALPCO Diagnostics, Salem, N.H.). Pancreatic tissues were dissected and fixed in 4% formaldehyde at 4° C. for 12 h before embedding in paraffin. Human and mouse 4-μm sections were deparaffinized, rehydrated and incubated overnight at 4° C. with anti-insulin (Dako), anti-P-MST1 (Cell Signaling), anti-Bim (Cell Signaling), anti-PDX-1 (abcam), anti-glucagon (Dako), anti-glut2 (Chemicon) and anti-mouse anti-Ki67 (BD Pharmingen) antibodies followed by fluorescein isothiocyanate (FITC)- or Cy3-conjugated secondary antibodies (Jackson ImmunoResearch Laboratories, West Grove, Pa.). Slides were mounted with Vectashield with 4′6-diamidino-2-phenylindole (DAPI) (Vector Labs). β-cell apoptosis for mouse sections or primary islets cultured on ECM dishes was analyzed by the terminal deoxynucleotidyl transferase-mediated dUTP nick-end labeling (TUNEL) technique according to the manufacturer's instructions (In Situ Cell Death Detection Kit, TMR red; Roche) and double stained for insulin. Fluorescence was analyzed using a Nikon MEA53200 (Nikon GmbH, Dusseldorf, Germany) microscope and images were acquired using NIS-Elements software (Nikon). For morphometric data, ten sections (spanning the width of the pancreas) per mouse were analyzed. Pancreatic tissue area and insulin-positive area were determined by computer-assisted measurements using a Nikon MEA53200 (Nikon GmbH, Dusseldorf, Germany) microscope and images were acquired using NIS-Elements software (Nikon). The number of islets (defined as insulin-positive aggregates at least 25 μm in diameter) was scored and used to calculate islet density (number of islets per square centimeter of tissue), mean islet size (the ratio of the total insulin-positive area to the total islet number on the sections). Mean percent β-cell fraction per pancreas was calculated as the ratio of insulin-positive and whole pancreatic tissue area. β-cell mass was obtained by multiplying the β-cell fraction by the weight of the pancreas. Morphometric β-cell and islet characterizations are results from analyses of at least 100 islets per mouse.

### Example 3

### Inhibition of MST1 Activity Improves β-Cell Survival and Function

To demonstrate that deficiency of MST1 activity improves β-cell survival and function, MST1 was depleted in human islets (FIG. 5). As a result, human islets protected from cytokine-, H₂O₂-, glucolipo-toxicity and β-cell apoptosis was inhibited. Silencing of MST1 also dramatically reduced Bim up-regulation induced by diabetogenic conditions in human islets. β-cell function was greatly improved by MST1 gene silencing under diabetogenic conditions. Notably, IL/IF- and HG/Pal-induced caspase-3 and -9 cleavage and P-H2B all decreased in MST1-depleted human islets. MST1^{-/-} islets largely resisted to IL/IF- and HG/Pal-mediated apoptosis as determined by TUNEL staining. In addition to its protective effect on β-cell survival, MST1^{-/-} islets also improved GSIS after long-term culture with IL/IF and HG/Pal. Human islets were isolated from twenty pancreata of healthy organ donors and from five with T2D at the University of Illinois at Chicago or Lille University and cultured on extracellular matrix (ECM) coated dishes (Novamed, Jerusalem, Israel) as described previously (Kurrer et al., PNAS 94 (1997), 213-218). Islet purity was greater than 95% as judged by dithizone staining (if this degree of purity was not achieved by routine isolation, islets were handpicked).

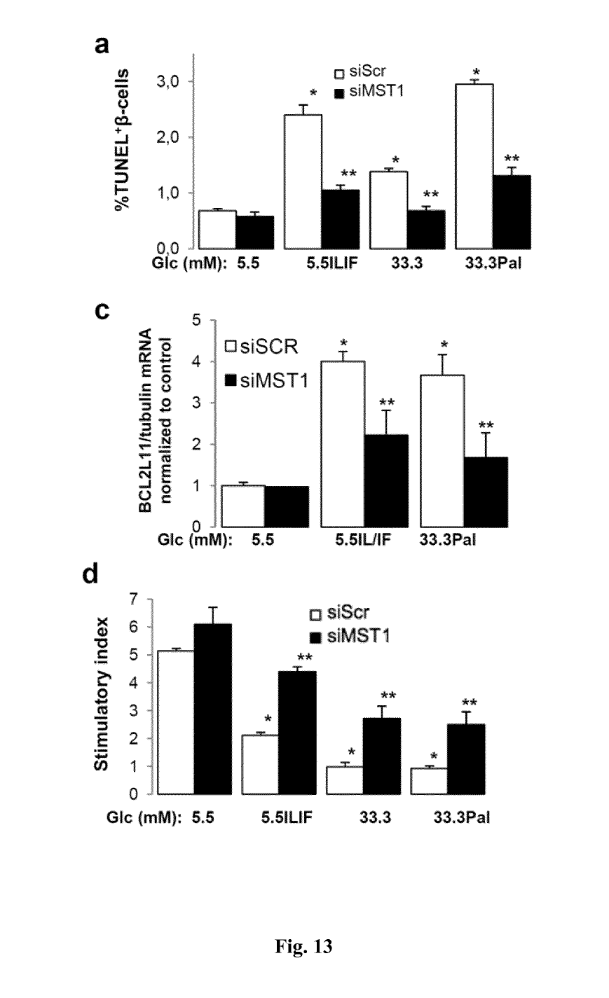

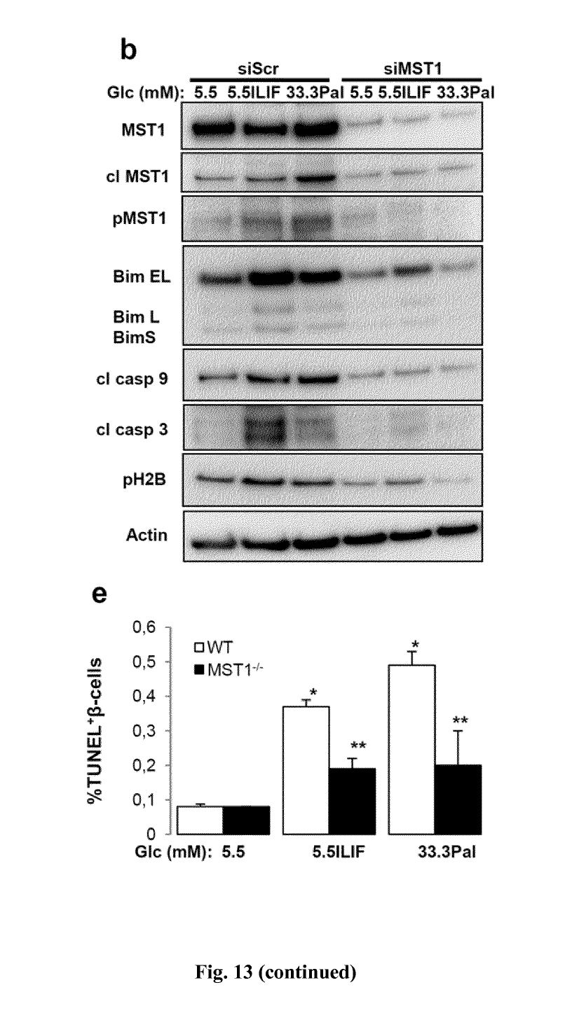

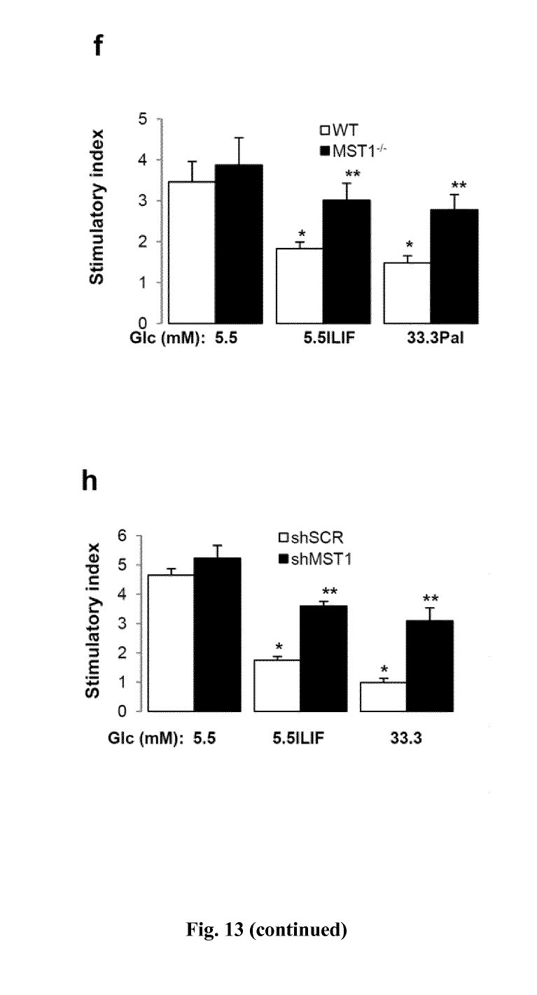

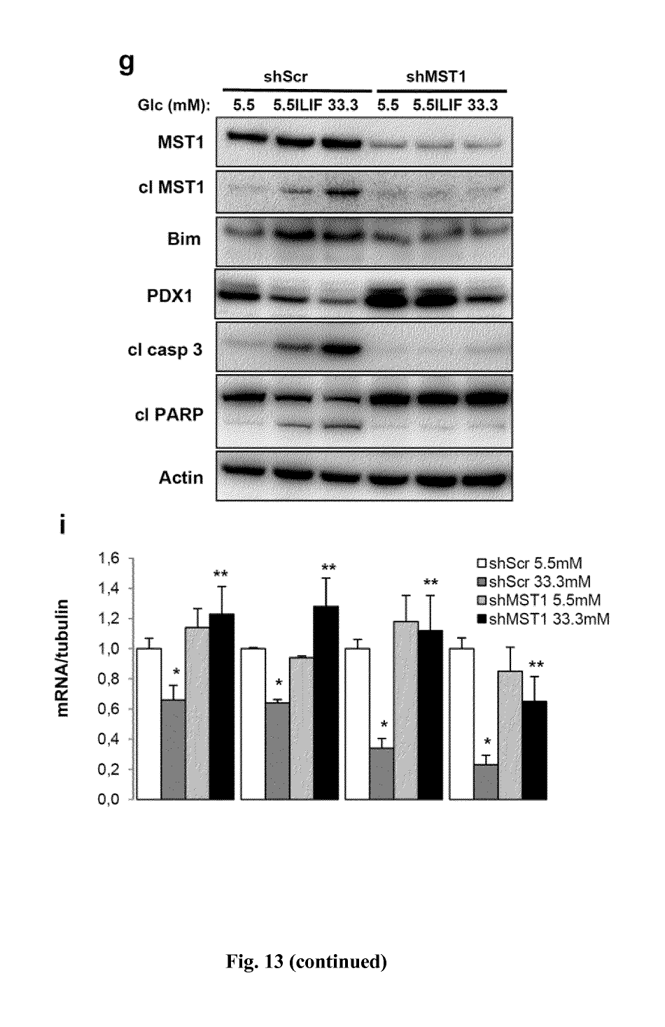

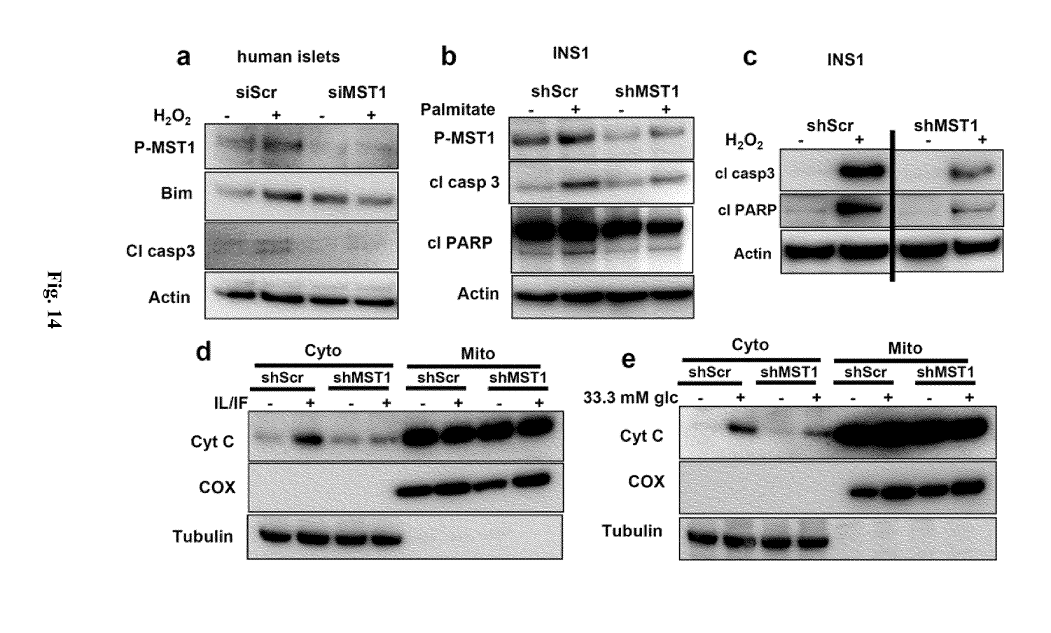

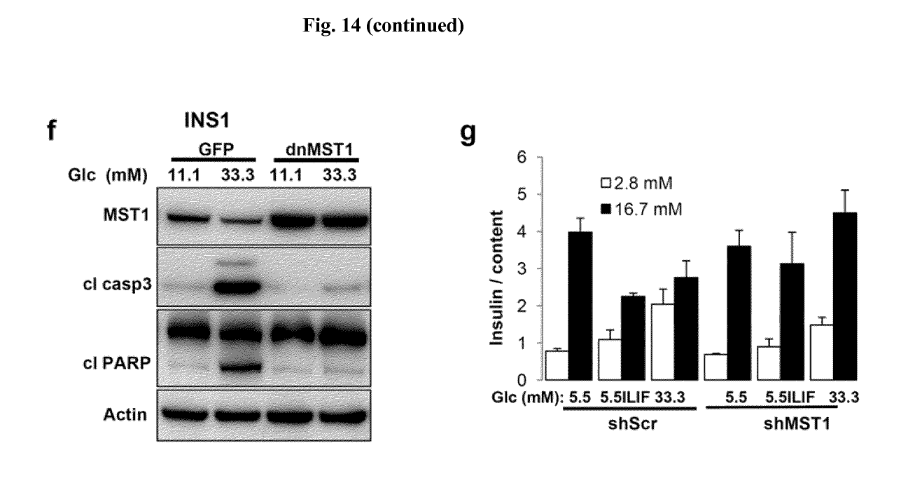

To knockdown MST1 in human islets, SMARTpool technology from Dharmacon was used as described, supra, in the Supplementary Methods and methods. To further support the role of MST1 as a main mediator of apoptosis in the β-cells, the clonal rat beta-cell line INS-1E cells were stably transfected with vector for shScr and shMST1 and the reduction in MST1 expression of the cells stably expressing shMST1 was confirmed (FIG. 14 g). To knock down MST1 expression in INS1E cells, lentiviral shRNAmir targeting MST1 or control shRNAmir vectors (pGIPZ collection, Open Biosystems, Huntsville, Ala.) were transfected into INS-1E cells and stable clones were generated by selection with puromycin (1 to 2.5 μg/ml). Positive clonal cell lines were identified by immunoblotting using antibody directed against MST1. After selection, INS1E lines were maintained in culture medium containing 1.5 μg/ml puromycin. INS1 clones were treated with IL/IF and HG for 72 h. Bim induction, caspase-3- and PARP-cleavage in MST1 depleted cells was markedly decrease compared to control cells (FIG. 13 g). Additionally, MST1 silencing also abrogated caspase-3 and PARP cleavage induced by palmitate (FIG. 13 b) and H₂O₂ (FIG. 13 c). Cytochrome c release was markedly reduced in MST1-depleted β-cells under diabetogenic conditions (FIG. 14 d, e). INS-1E cells were cultured in complete RPMI-1640 medium at 11.1 mM glucose. Media included with glutamate, 1% penicillin-streptomycin and 10% fetal bovine serum (FBS, all PAA). INS-1E medium was supplemented with 10 mM HEPES, 1 mM sodium pyruvate and 50 μM β-mercaptoethanol. INS-1E were exposed to complex diabetogenic conditions: 22.2-33.3 mM glucose, 0.5 mM palmitic acid, the mixture of 2 ng/ml recombinant human IL-1β (R&D Systems, Minneapolis, Minn.)+1,000 U/ml recombinant human IFN-γ (PeProTech) for 72 h, 100 μM H₂O₂ for 6 h, 1 mM STZ) for 8 h or 1 mM thapsigargin for 6 h (all Sigma).

In confirmation with the shMST1 approach, it was shown that inhibition of endogenous MST1 activity by overexpression of dnMST1 completely inhibited glucose-induced caspase-3 and PARP cleavage in β-cells (FIG. 14 f). Notably, MST1 deficiency prevented PDX1 depletion upon cytokine and high glucose treatment, implying that MST1 is indispensable for the PDX1 reduction induced by a diabetic milieu (FIG. 13 g). The next objective was to determine whether MST1 knockdown leads to improvement of GSIS and restoration of PDX1 target genes in INS-1E cells under diabetogenic conditions. The significant reduction in the mRNA level of PDX1 target genes, e.g. SLC2A2, GCK, Ins1 and Ins2 was prevented and GSIS significantly improved in MST1 depleted β-cells (FIG. 13 h, i and FIG. 14 g). These data suggest MST1 as determinant for β-cell apoptosis and defective insulin secretion under a diabetic milieu in β-cells in vitro.

### Example 4

### MST1 Impairs β-Cell Function Through Destabilization of PDX1

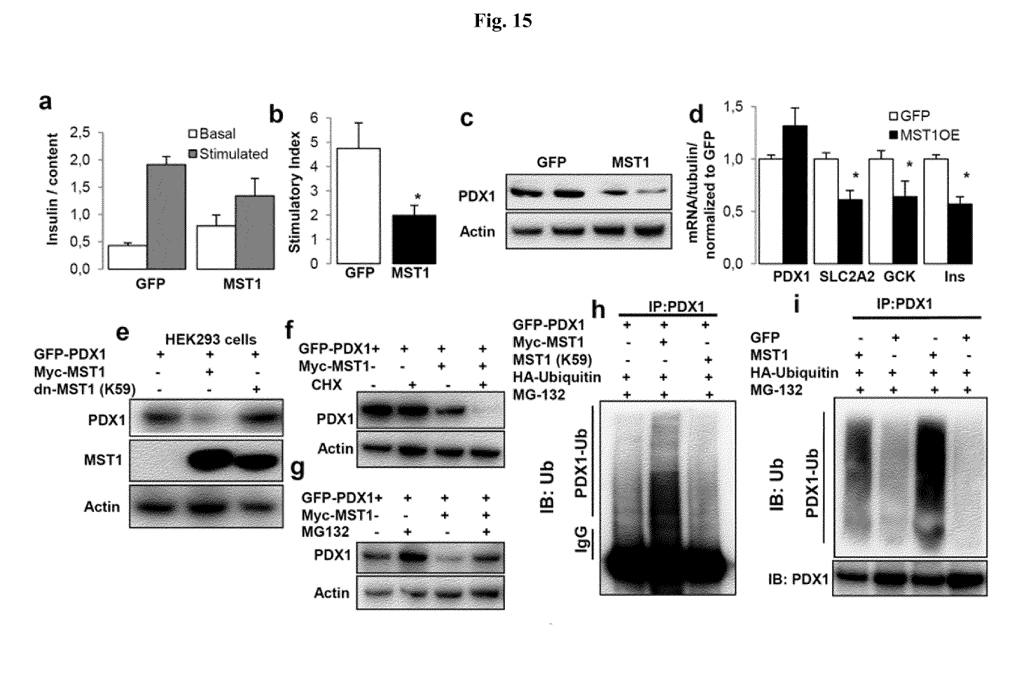

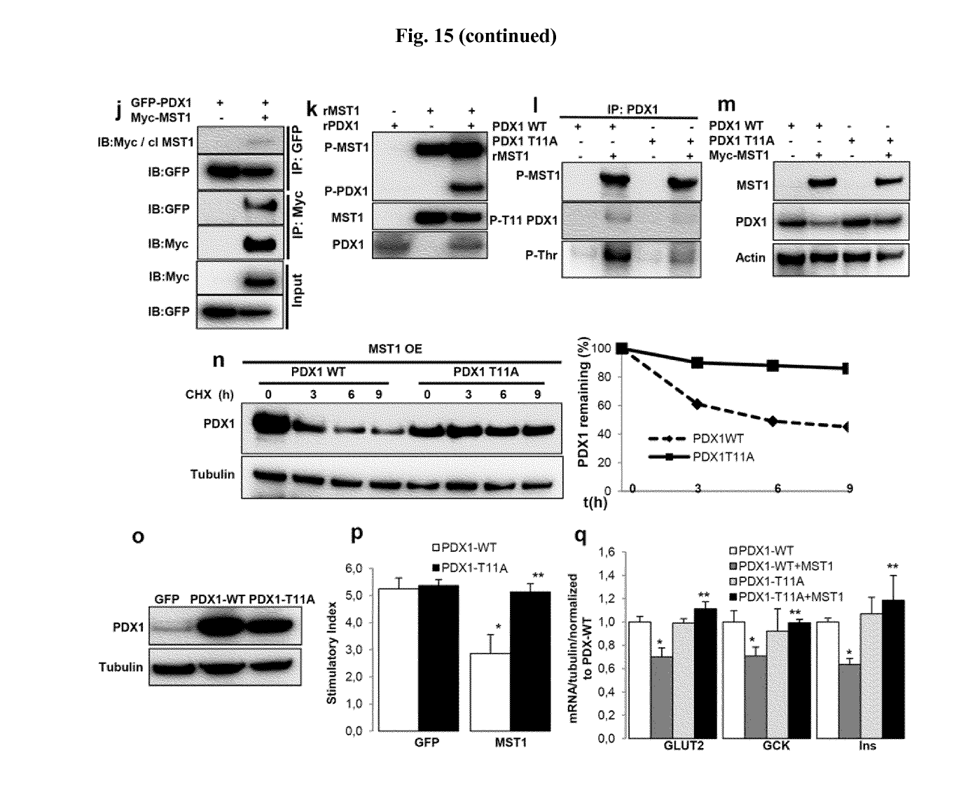

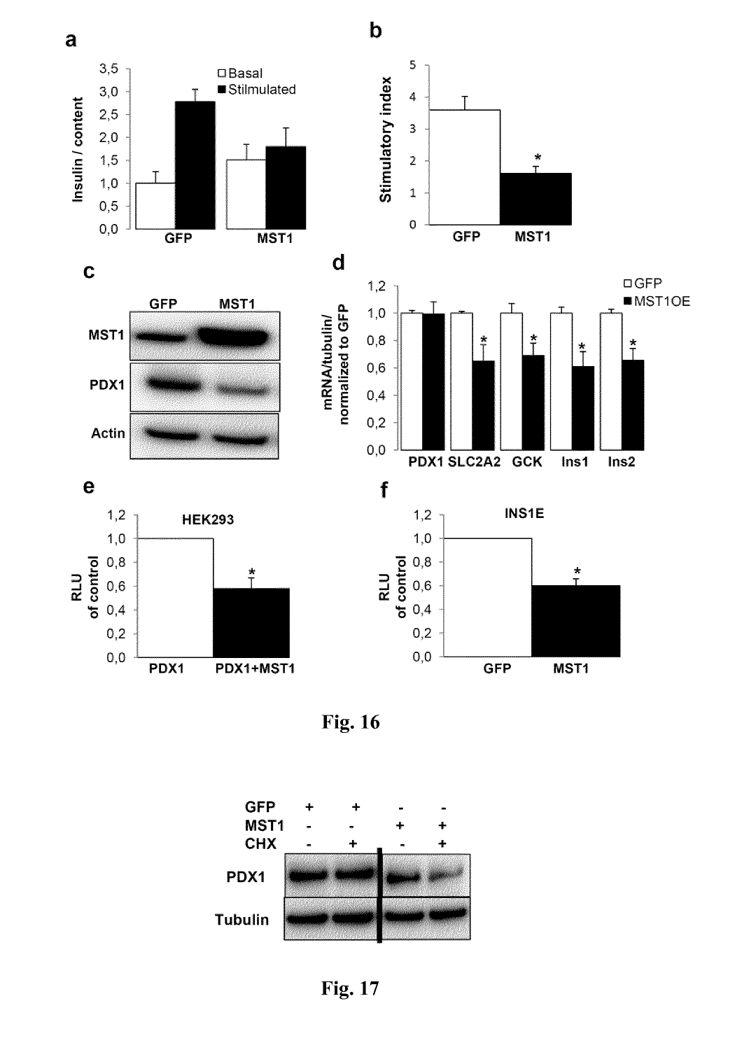

It was hypothesized that MST1 activation may elicit changes in β-cell specific gene transcription that initiate the process of β-cell failure. Overexpression of MST1 led to a complete loss of glucose-stimulated insulin secretion (GSIS; FIG. 15 a-b and FIG. 16 a-b), which could not be accounted solely by the induction of apoptosis. Previously, it was noted that the critical β-cell PDX1) which mediates glucose-induced insulin gene transcription in mature β-cells is mislocalized and reduced in diabetes. These changes are subsequently associated with impaired β-cell function and hyperglycemia. Stress-induced kinases such as JNK and glycogen synthase kinase-3 (GSK3) phosphorylate and antagonize PDX1 activity leading to β-cell failure. Thus, it was hypothesized that the drastic reduction in insulin secretion following MST1 overexpression may be mediated by PDX1. PDX1 levels were markedly reduced in response to MST1 overexpression in human islets (FIG. 14 c) and INS-1E cells (FIG. 16 c). In contrast, MST1 overexpression did not affect PDX1 mRNA levels (FIG. 15 d and FIG. 16 d), suggesting that MST1 may regulate PDX1 at the post-transcriptional level.

Real time PCR analysis of PDX1 target genes demonstrated that overexpression of MST1 significantly down-regulated Insulin (Ins1 or Ins2 for INS-1E), SLC2A2 and GCK in human islets (FIG. 15 d) and INS-1E cells (FIG. 16 d). Total RNA was isolated from cultured human islets and INS1 cells using TRIzol (Invitrogen), and RT-PCR performed as described previously. For analysis, we used the Applied Biosystems StepOne Real-Time PCR system (Applied Biosystems, CA, USA) with TaqMan® Fast Universal PCR Master Mix for TaqMan assays (Applied Biosystems). TaqMan® Gene Expression Assays were used for pdx1 (Hs00426216_m1), SLC2A2 (Hs01096905_m1), GCK (Hs01564555_m1), insulin (Hs02741908_m1), PPIA (Hs99999904_m1) and tubulin (Hs00362387_m1) for human and PDX1 (Rn00755591_m1), SLC2A2 (Rn00563565_m1), GCK (Rn00688285_m1), INS1 (Rn02121433_g1), INS2 (Rn01774648_g1), BCL2L11 (Hs01083836_m1), PPIA (Rn00690933_m1) and tuba1a (Rn01532518_g1) for rat. To clarify the mechanism by which MST1 regulates PDX1, the effects of ectopic expression of MST1 and PDX1 were examined in HEK293 cells. This revealed decreased PDX1 level in cells co-overexpressing MST1, whereas a kinase-dead MST1 (K59R; dnMST136) had no effect (FIG. 15 e). Thus, kinase activity is required for MST1-induced PDX1 degradation.

Overexpression of MST1 also attenuated the transcriptional activity of PDX1 on the rat insulin promoter, as shown by luciferase assays in HEK293-overexpressing PDX1 (FIG. 16 e) and INS-1E cells (FIG. 16 f). The transcriptional activity of the PDX1 at promoter level was evaluated using rat Ins2-Luc renilla reporter gene HEK293 cells were transfected with Ins2-Luc renilla, pCMV-firefly, PDX1-WT or PDX1-T11A, alone or together with Myc-MST1 expressing plasmids for 48 h. INS-1E cells transfected with Ins2-Luc renilla and pCMV-firefly plasmids and were infected with Ad-GFP or Ad-MST1 for 48 h. Luciferase activity determined using the Renilla-Firefly Luciferase Dual Assay Kit according to the manufacturer's instructions (Pierce). pCMV-firefly was used as transfection control. To discriminate between a transcriptional/translational and a post-translational effect of MST1 on PDX1, we followed the stability of overexpressed PDX1 upon treatment with cycloheximide (CHX), an inhibitor of protein translation. PDX1 protein levels rapidly decreased when co-expressed with MST1 upon CHX exposure (FIG. 15 f), which suggests that MST1 reduced PDX1 protein stability. Consistent with these observations, MST1 overexpression also decreased protein stability of endogenous PDX1 in human islets (FIG. 17). In contrast, inhibition of proteasomal degradation by treatment of PDX1 overexpressing HEK293 cells with the proteasome inhibitor MG-132 abolished the disappearance of PDX1 (FIG. 15 g), indicating that MST1-induced activation of the ubiquitin proteasome pathway. Proteasomal degradation of PDX1 has been described before and leads to impaired β-cell function and survival. In vivo ubiquitination assays were next performed to determine whether MST1 induces PDX1 ubiquitination. PDX1 co-transfected with MST1 but not with MST1-K59 was heavily ubiquitinated in HEK293 cells (FIG. 15 h). This was confirmed in human islets by showing that MST1 overexpression strongly promoted endogenous PDX1 ubiquitination (FIG. 15 i). Subsequently, a direct interaction between PDX1 and MST1 proteins were verified. Reciprocal co-immunoprecipitations showed the interaction between MST1 and PDX1 in HEK293 cells co-transfected with GFP-tagged PDX1 and myc-tagged MST1 (FIG. 15 j).

### Example 5

### Phosphorylation Mutant of PDX1 Antagonizes MST1 Activity and Restores β-Cell Function

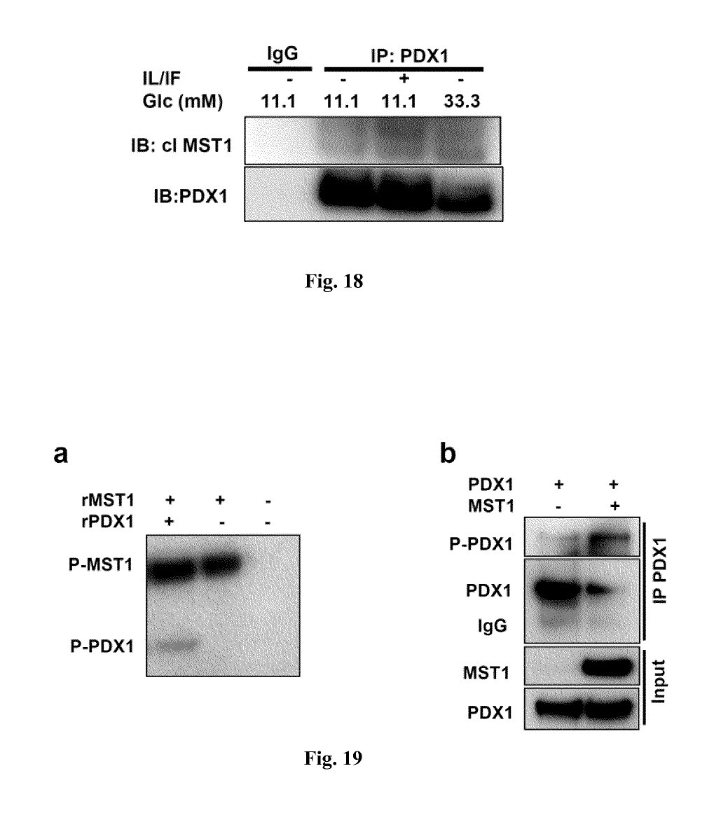

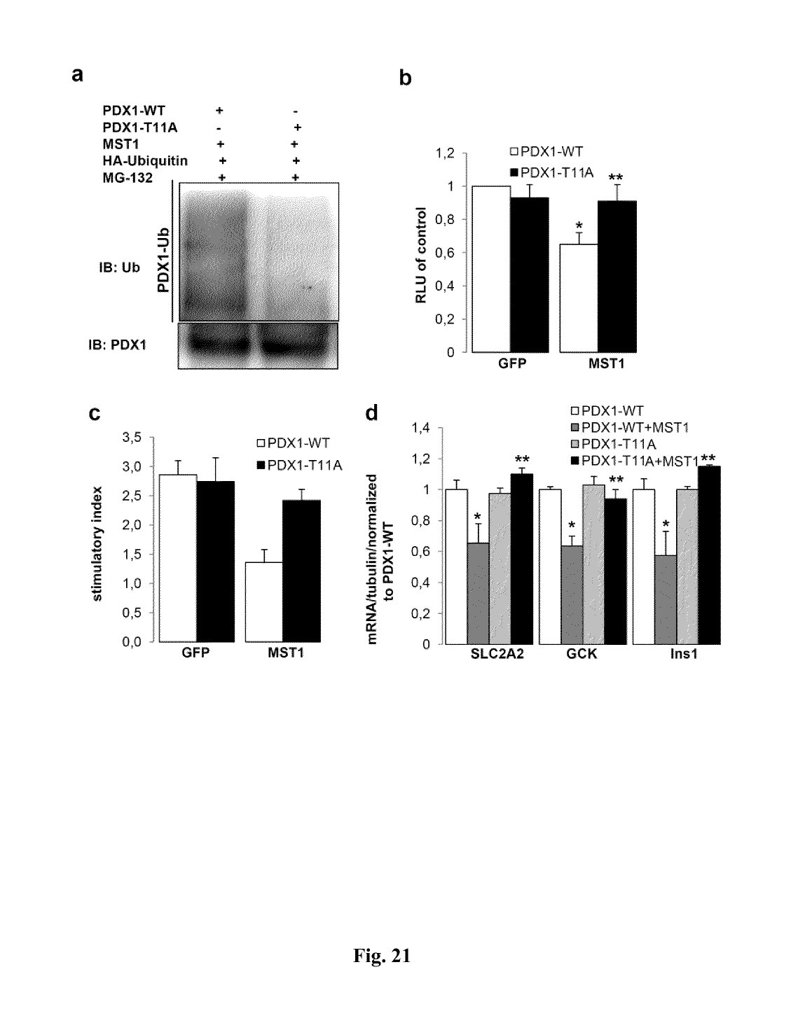

Furthermore, it was examined whether a pro-diabetic milieu regulates the association between MST1 and PDX1. Strikingly, both cytokine- and glucotoxicity increased the interaction between MST1 and PDX1 in INS-1E cells (FIG. 18). Since it was observed that PDX1 ubiqutination and degradation required MST1 kinase activity, it was tested whether MST1 directly phosphorylates PDX1. In vitro kinase assays showed that MST1 efficiently phosphorylated PDX1 shown by autoradiography using radio labeled ³²P (FIG. 19 a) and by non-radioactive kinase assays and western blotting using a phospho-specific PDX1 antibody (FIG. 15 k). The in vitro kinase assays were confirmed in HEK293 cells; co-expression of MST1 and PDX1 led to PDX1 phosphorylation (FIG. 19 b). Together, these results establish PDX1 as a substrate for MST1. The potential MST1-targeted phosphorylation sites of PDX1 were determined theoretically with the Netphos 2.0 program. This identified six candidate sites including T11, T126, T152, T155, T214 and T231 within the PDX1 sequence based on a relative score (FIG. 20 a). These 6 sites were individually mutated to alanine to generate phospho-deficient constructs (Frogne et al., PLoS One 7 (2012) e35233). PDX1-GST fusion proteins with different PDX1 mutations were purified from bacteria and used as substrates for MST1 in the kinase assay. With the exception of T11A, PDX1-WT and other mutants were efficiently threonine phosphorylated (FIG. 20 b). To confirm this, all PDX1 mutants were transfected into HEK293 cells, immunoprecipitated with PDX1 and incubated with recombinant MST1 in a kinase assay. MST1 highly phosphorylated WT-PDX1 and other mutants, while phosphorylation in the PDX1-T11A was markedly decreased, indicating that T11 is major site of phosphorylation by MST1. To further validate this, a phosphospecific antibody against T11 phosphorylation site in PDX1 (p-T11PDX1) recognized wild-type recombinant PDX1, which was incubated with MST1 in the kinase assay (FIG. 20 c). Consistently, co-incubation of immunoprecipitated PDX1-WT or PDX1-T11A with recombinant MST1 resulted in robust MST1-induced PDX1-WT phosphorylation at the Thr11 site (shown by p-T11 antibody) and in overall Thr-phosphorylation (shown by pan-Threonine antibody); such phosphorylation was markedly reduced in the PDX1-T11A mutant protein (FIG. 15 i). This was further corroborated by an in vivo kinase assay (FIG. 20 d). Alignment of the amino acid sequences of PDX1 from different species revealed that Thr11 site is highly conserved among those species (FIG. 20 e). If Thr11 is the specific MST1-induced phosphorylation site of PDX1 and responsible for β-cell dysfunction, one would expect that mutated PDX1-T11A would reverse such deleterious effects of MST1. This hypothesis was supported by the observation that MST1 does not decrease PDX1 levels in PDX1-T11A-expressing HEK293 cells (FIG. 15 m). MST1 induced a rapid degradation of PDX1 in the presence of CHX, which did not occur in PDX1-T11A mutant transfected cells (FIG. 15 n). Furthermore, the half-life of the PDX1-T11A mutant was similar as PDX1-WT in the absence of MST1. Consistently, there was less PDX1 ubiquitination in the PDX1-T11A-transfected cells than in PDX1-WT (FIG. 21 a). Since Thr11 is located within the transactivational domain of PDX1 and to evaluate the functional significance of the Thr11-dependent ubiquitination/degradation, transcriptional activity of PDX1 was assessed. Reduction of PDX1 transcriptional activity occurred only in PDX1-WT but not in PDX1-T11A mutant transfected cells (FIG. 21 b). Since mutation of PDX1 on Thr11 maintains PDX1 stability, it was hypothesized whether PDX1 stability is directly linked to improved β-cell function. Indeed, PDX1-T11A mutant overexpression (FIG. 15 o) normalized MST1-induced impairment in GSIS in human islets (FIG. 15 p) and INS-1E cells (FIG. 21 c) and restored MST1-induced down regulation of PDX1 target genes (FIG. 15 q and FIG. 21 d). These findings indicate that MST1-induced PDX1 phosphorylation at Thr11 leads directly to PDX1 de-stabilization and impaired β-cell function and suggest that PDX1 is a crucial target of MST1 in the regulation of β-cell function.

### Example 6

### MST1 Induces β-Cell Death Through Activation of the Mitochondrial Apoptotic Pathway

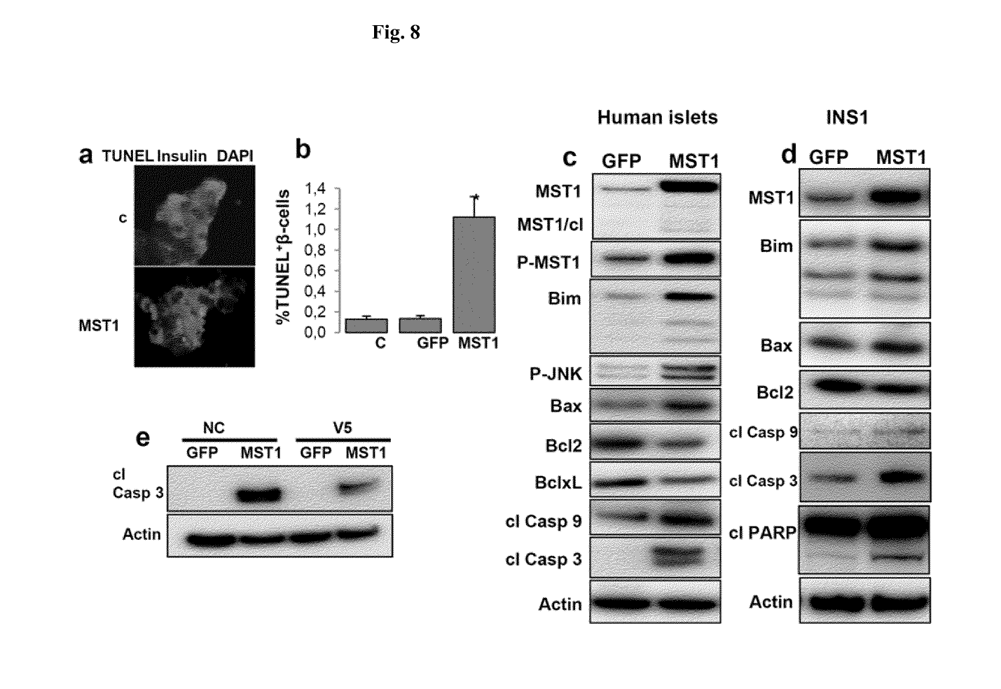

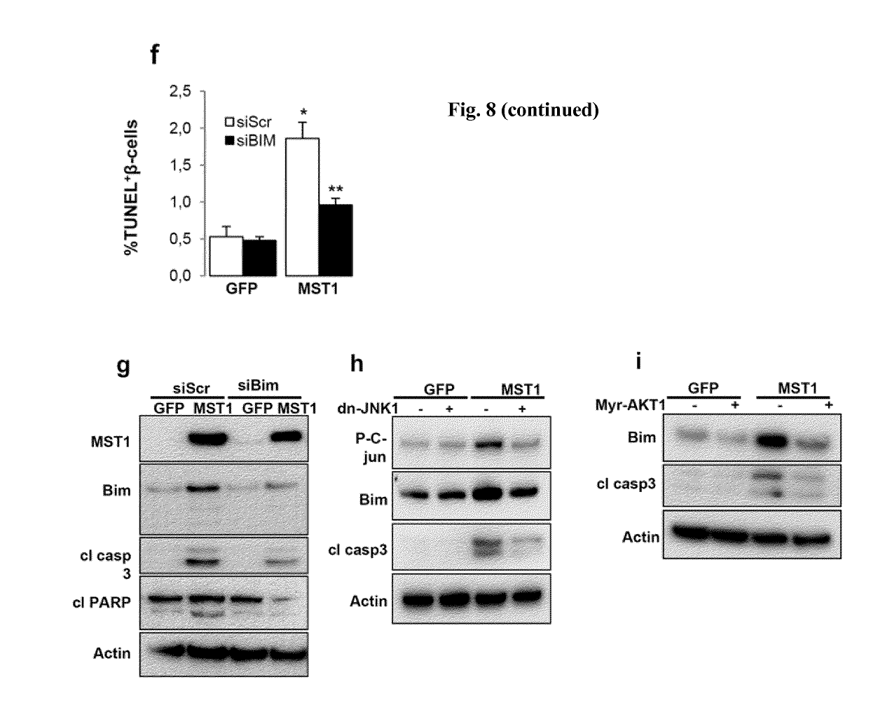

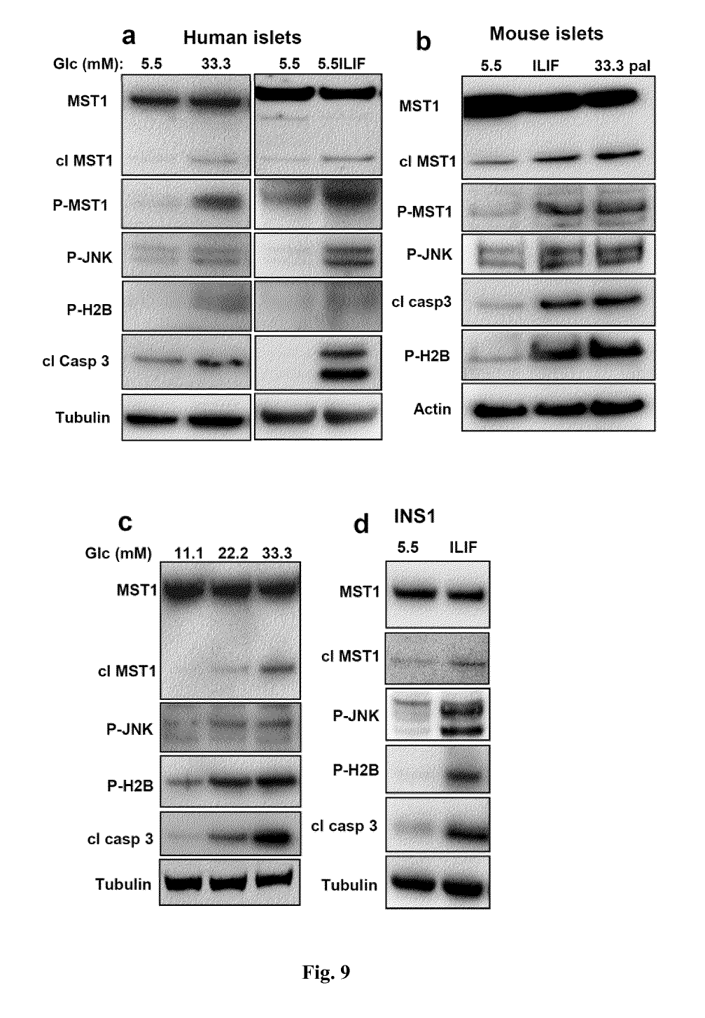

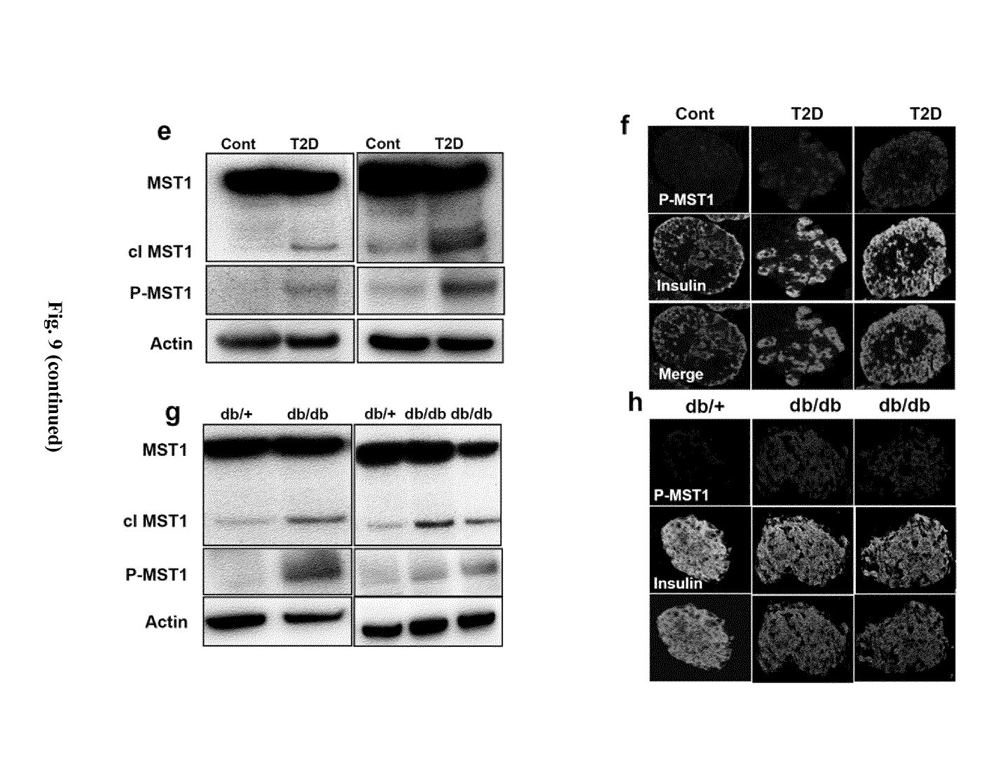

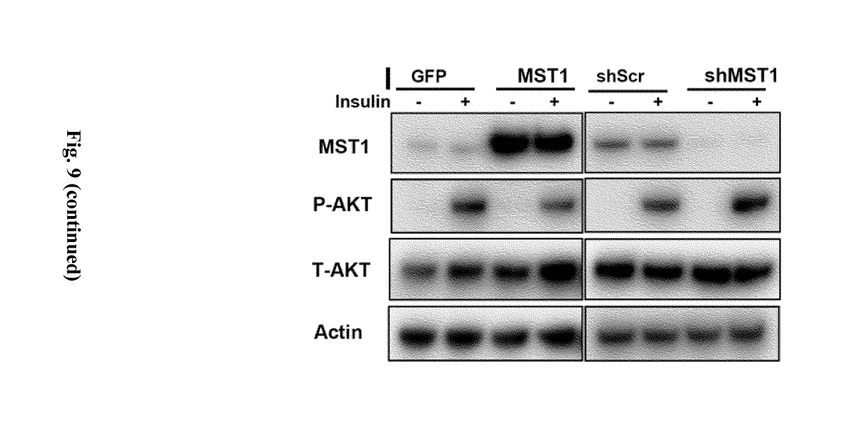

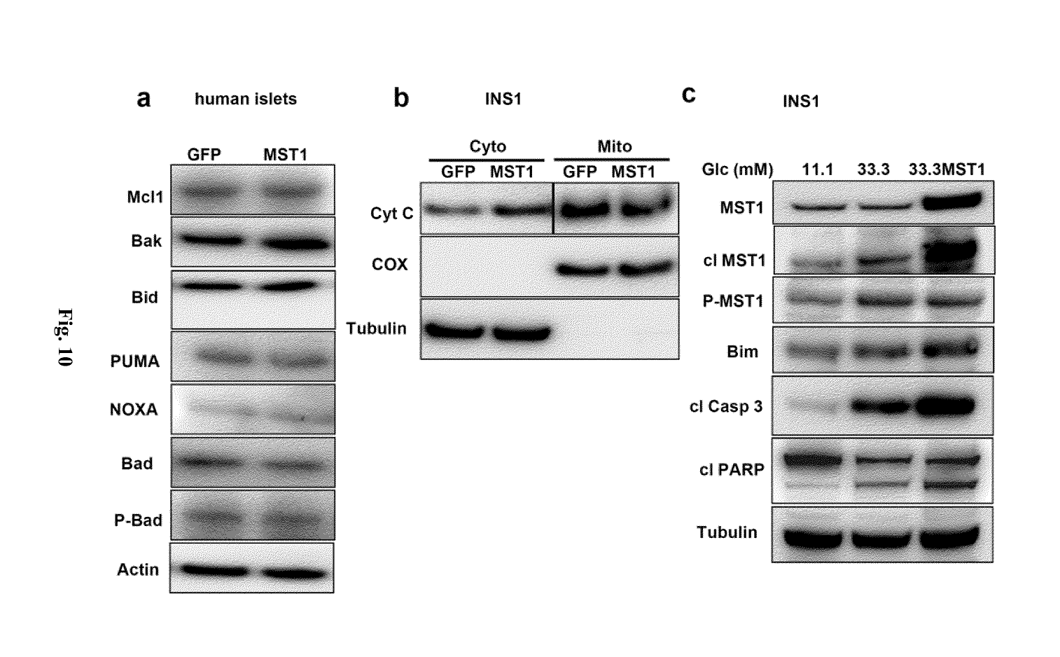

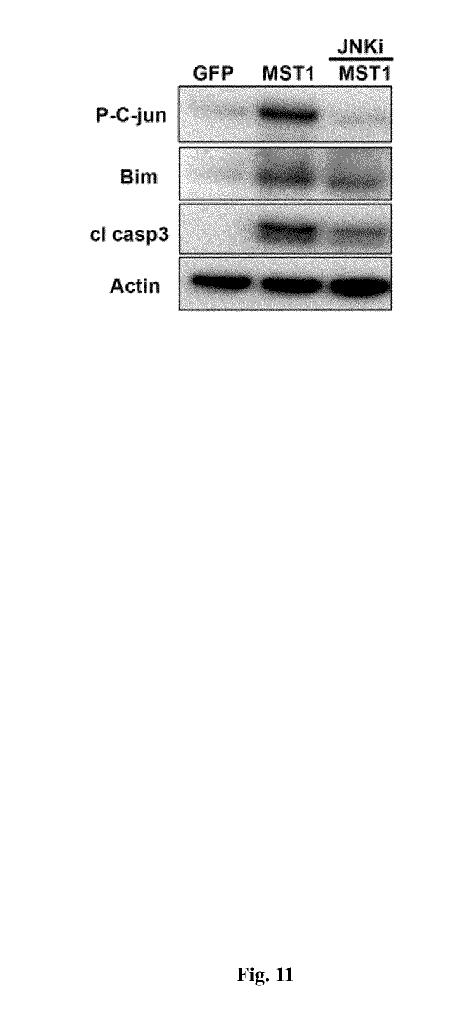

To investigate pathways that potentially contribute to MST1-induced β-cell apoptosis, MST1 was overexpressed in human islets and INS-1E cells through an adenoviral system, which efficiently up-regulated MST1, increased number of TUNEL-positive β-cells and activated JNK, PARP- and caspase-3 cleavage (FIG. 8 a-d). Previous data proposed a role of the mitochondrial pathway in MST-dependent signaling. Profiling expression levels of established mitochondrial proteins in MST1-overexpressing islets showed cleavage of the initiator caspase-9, release of cytochrome c, induction of pro-apoptotic Bax and a decline in anti-apoptotic Bcl-2 and Bcl-xL levels (FIG. 8 c-d and FIG. 4 b), which led to a reduction of Bcl-2/Bax and Bcl-xL/Bax. Notably, MST1-induced caspase-3 cleavage was reduced by treatment of human islets with the Bax-inhibitory peptide V5 (FIG. 9 e), which was shown to promote β-cell survival and emphasizes that MST1-induced apoptosis proceeds via the mitochondrial-dependent pathway. The expression was analyzed of BH3-only proteins as regulators of the intrinsic cell death pathway. Of these, Bim was robustly induced, whereas other BH3-only proteins levels remained unchanged (FIG. 8 c-d and FIG. 10 a). It was determined whether Bim is a major molecule to take over the pro-apoptotic action of MST1. Indeed, Bim depletion led to a significant reduction of MST1-induced apoptosis in human islets (FIG. 8 f, g). Overexpression of MST1 further potentiated glucose-induced apoptosis in β-cells in a Bim-dependent manner (FIG. 10 c). Bim is regulated by the JNK and AKT signaling pathways. MST1-induced increase in Bim and subsequent caspase-3 cleavage was prevented by JNK inhibition using two strategies; overexpression of dnJNK1 (FIG. 8 h) and pharmacological JNK inhibition (FIG. 11) suggesting that MST1 uses JNK signaling to mediate Bim up-regulation and induction of apoptosis. The involvement of AKT in the regulation of MST1-induced apoptosis was confirmed by co-overexpression of MST1 and Myr-AKT1, which reduced Bim induction and caspase-3 cleavage (FIG. 8 i), indicating that AKT negatively regulates the downstream target of MST1. These data show that MST1 is a critical mediator of β-cell apoptosis through activation of the Bim-dependent intrinsic apoptotic pathway and controlled by AKT- and JNK signaling pathways.

### Example 7

### MST1 is Activated by Diabetogenic Conditions and Correlates with β-Cell Apoptosis

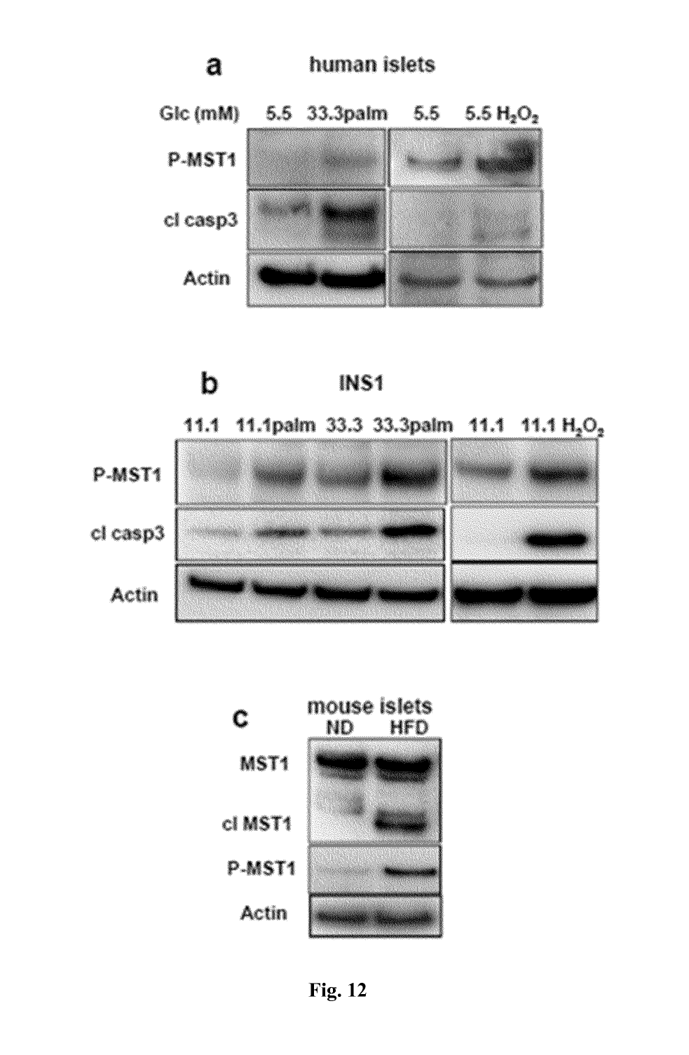

To identify MST1 activation and its correlation with β-cell apoptosis, isolated human and mouse islets and the β-cell line INS-1E were exposed to a complex diabetic milieu in vitro (cytokine mixture IL-1β/IFNγ:IL/IF, increasing glucose concentrations, palmitic acid and oxidative stress: H₂O₂). MST1 was highly up-regulated by all diabetic conditions (FIG. 9 a-d and FIG. 12 a, b) in β-cells, which occurred by both caspase-mediated cleavage and through auto-phosphorylation (P-MST1-T183). This was accompanied by increased phosphorylation of histone H2B as well as induction of c-jun N-terminal kinase (JNK) signaling (FIG. 9 a-d). MST1 was also activated in islets from T2D patients (FIG. 8 e, f), obese diabetic Lepr^{db/db} mice (db/db, FIG. 9 g, h) and from hyperglycemic HFD mice for 16 weeks (Surwit, FIG. 12 c), which correlated with β-cell apoptosis as described before. To confirm the β-cell specific up-regulation of MST1, double-staining for P-MST1 and insulin in pancreatic islets from poorly controlled patients with T2D (FIG. 9 f) as well as db/db mice (FIG. 9 h) showed expression of P-MST1 in β-cells, while no signal was observed in non-diabetic patients and control mice.

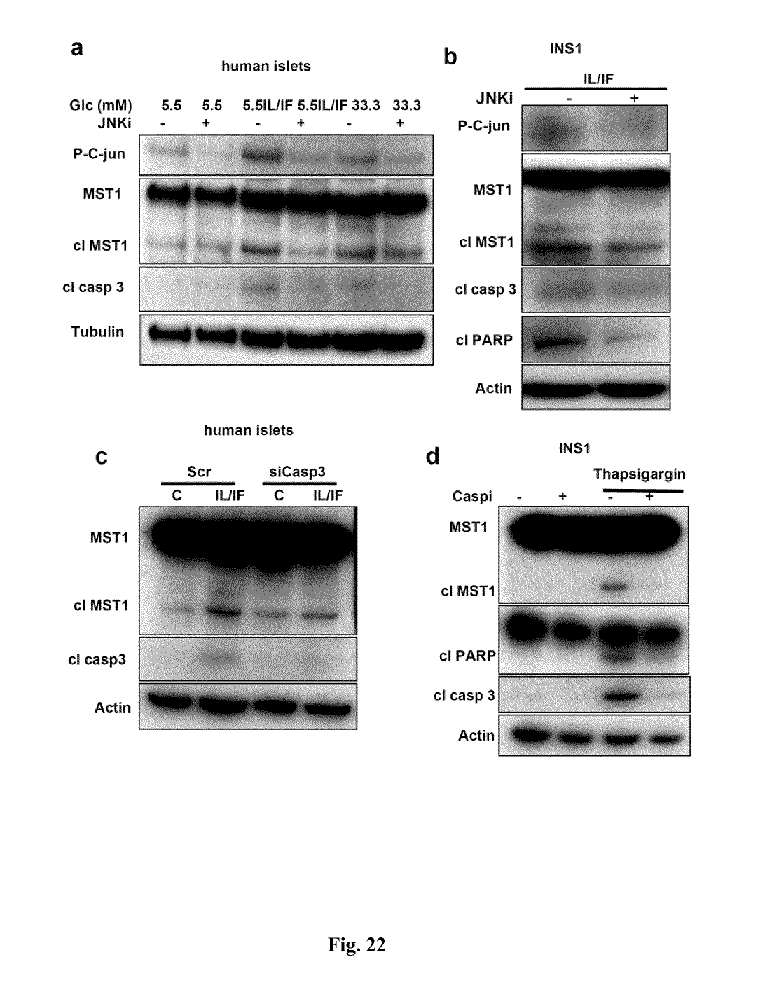

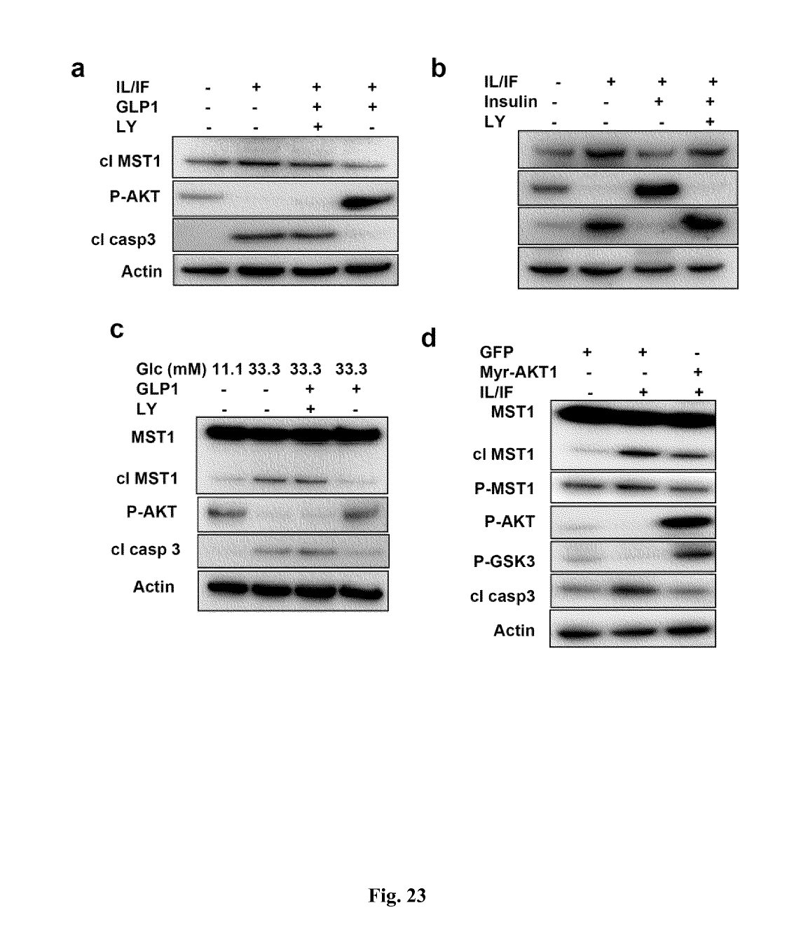

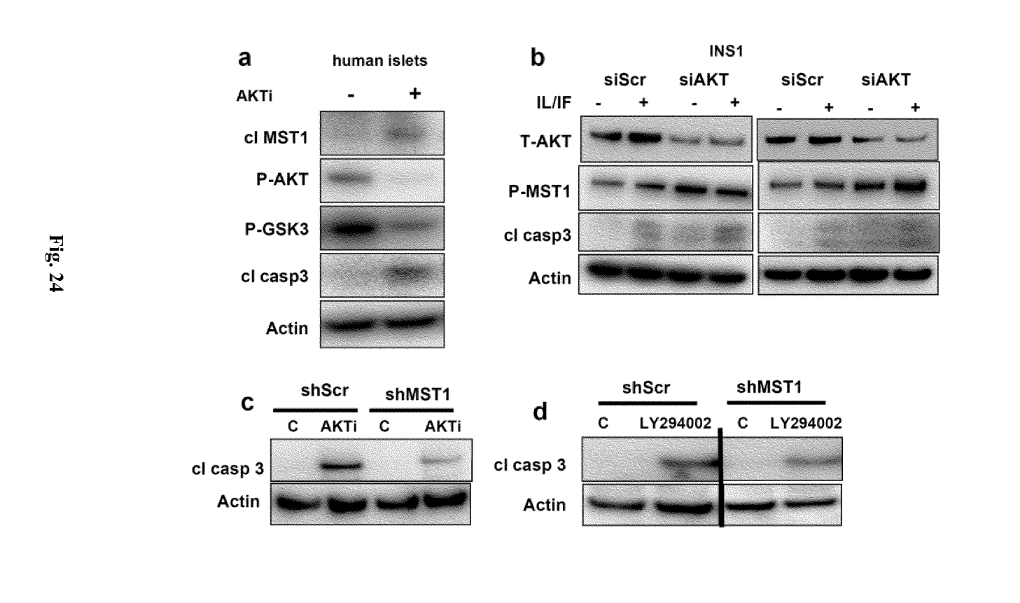

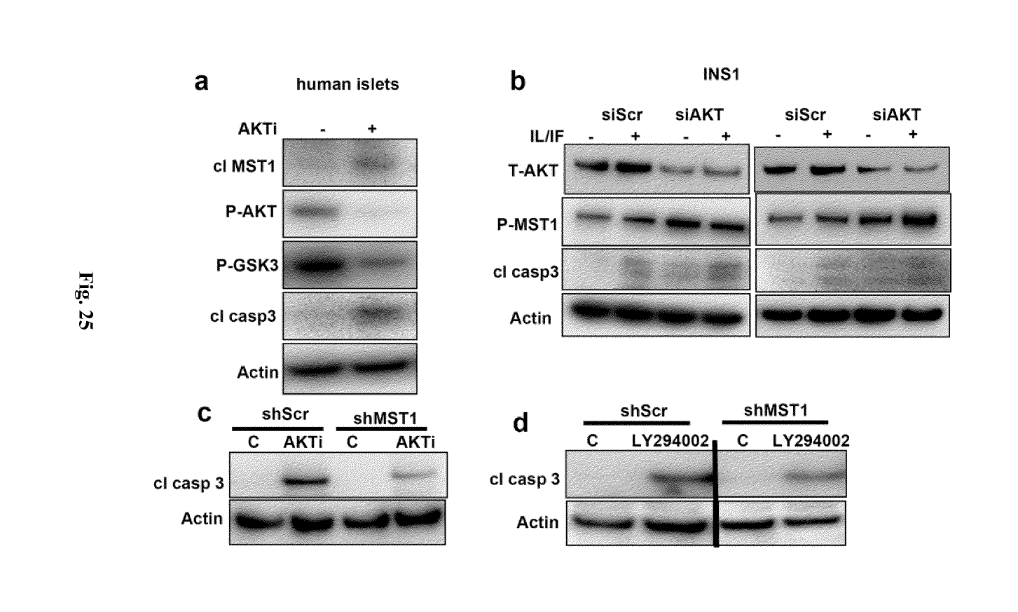

Caspase-3 and JNK act not only as downstream targets, but also as upstream activators of MST1 through cleavage- and phosphorylation-dependent mechanisms and may initiate a vicious cycle and a pro-apoptotic signaling cascade in the β-cell. Using JNK (SP600125) and caspase (z-DEVD-fmk) inhibitors and siRNA to caspase-3 (siCasp3), it was found that both JNK and caspase-3 were responsible for stress-induced MST1 cleavage by diabetic stimuli in human islets and INS-1E cells (FIG. 22 a-d), suggesting that MST1 induces a positive feedback loop with caspase-3 under diabetogenic conditions. Because phosphatidylinositol-3 kinase (PI3K)/AKT signaling is a key regulator of β-cell survival and function and since MST1 signaling is negatively regulated by this pathway in other cell types, we hypothesized that AKT is an important negative regulator of MST1. Maintaining AKT-activation through either exogenously added mitogens like GLP1 or insulin or overexpression of constitutively active AKT1 (Myr-AKT1) inhibited glucose- and cytokine-induced P-MST1, MST1-cleavage and apoptosis (FIG. 9 i and FIG. 23). Since GLP-1 and insulin exert their cell survival actions primarily through the PI3K/AKT pathway, it was tested whether inhibition of this pro-survival signaling might enhance MST1 activation. PI3K and AKT were inhibited by LY294002 and triciribine (AKT inhibitor) led to decreased levels of phosphorylation of GSK3 and FOXO1, two well-characterized AKT substrates and induced MST1 activation (FIG. 9 j, k and FIG. 24 a). This was further corroborated using siRNA against AKT, which led to a critical up-regulation of MST1 activity and potentiated cytokine-induced P-MST1 and β-cell death (FIG. 24 b), also shown by diminished insulin-induced AKT phosphorylation in the presence of MST1; and conversely by enhanced AKT phosphorylation in MST1-depleted β-cells (FIG. 9 l). Knockdown of MST1 antagonized the apoptotic effect of AKT inactivation in INS-1E cells, implicating endogenous MST1 in the apoptotic mechanism induced by PI3K/AKT inhibition (FIG. 24 c, d). In summary, these results suggest that MST1 is activated in pro-diabetic conditions in vitro and in vivo, that is antagonized by PI3K/AKT signaling (FIG. 25) and depends on the JNK- and caspase-induced apoptotic machinery.

### Example 8

### Validating MST1 Antagonists In Vitro/In Vivo for their Efficiency to Restore β-Cell Survival and/or to Reverse Diabetes

As mentioned, in the description herein-above, in one embodiment the MST1 antagonist may be peptide kinase inhibitor derived from PDX1 comprising phosphorylation site Thr11. Corresponding peptides, preferably 12 to 22 amino acids in length are examined for phosphorylation by MST1 in an in vitro kinase assay as described in Example 5 using a phosphospecific antibody against T11 phosphorylation site in PDX1 (p-T11PDX1) for identifying and select those peptides which are most efficiently phosphorylated.

Candidate peptides are then tested whether they are capable of interfering with MST1 phosphorylation of native PDX1 and MST1 mediated decreased protein stability of PDX1 as described in Example 4, using for example the PDX1Thr11A mutant as a positive control.

Peptide kinase inhibitors so identified are then assayed in rodent and human islets and β-cell lines which are pre-treated with the peptide and exposed to diabetogenic conditions in culture as described in Example 5. Candidate peptides which give similar results like the PDX1Thr11A mutant, i.e. being capable of normalizing MST1-induced impairment in GSIS in human islets as well as INS-1E cells, and which are able to restore MST1-induced down regulation of PDX1 target genes are further investigated in vivo. Thus, animal models of T1D and T2D such as described in Examples 1 to 3 (BB rat, NOD mouse, MLD-STZ mouse and rat, STZ mouse and rat, HFD fed mice and rats, db/db mouse, ZDF rat, VDF rat, NZO mouse) are injected with the MST1 peptide kinase inhibitor. Glycemia, glucose tolerance, insulin tolerance and insulin secretion is monitored frequently and β-cell mass and survival and MST1 activation in pancreatic islets is analyzed at the end of the therapeutic period. Candidate peptides which show similar phenotypic effects as observed in the β-specific MST1^{-/-} mouse model used as a positive control are selected for clinical trials

Alternatively, or in addition for in vivo applications, the peptide kinase inhibitor is covalently linked to the 10-amino acid HIV-TAT sequence that directs cellular import in cells and animals. Corresponding peptides which may be chemically synthesized are expected to display improved cell-permeability and penetrate β-cells throughout the cytoplasm and the nucleus. Furthermore, the peptide kinase inhibitor may be synthesized in the all-D retro-inverso form that conserves all of the essential biological properties of the L-enantiomer and typically has a markedly expanded half-life in vivo. A corresponding approach has been described for cell-permeable peptide inhibitors of JNK in order to block cell death in diabetes; see, e.g., Bonny et al., Diabetes 50 (2001), 1 77-182, the disclosure content of which is incorporated herein by reference.

1. A method of treating or preventing a metabolic disease in an individual comprising administering an effective amount of a Mammalian Sterile 20-like kinase (MST) 1 antagonist to an individual in need thereof.

2. The method according to claim 1, wherein the metabolic disease is selected form the group consisting of diabetes and diabetes related diseases.

3. The method according to claim 1 wherein the MST1 antagonist is administered to treat one of type 1 diabetes (T1D) and type 2 diabetes (T2D), or to prevent progressive hyperglycemia or to improve glucose tolerance.

4. The method according to claim 1, wherein the MST1 antagonist is capable of reducing or inhibiting the binding of MST1 to Pancreatic and duodenal homeobox (PDX) 1 and/or phosphorylation of PDX1 by MST1 at amino acid site threonine (Thr) 11.

5. The method according to claim 1, which wherein the MST1 antagonist is an antibody, siRNA, shRNA, a kinase inhibitor, or a dominant mutant of MST1 (dnMST1) or a mutant PDX1 wherein the phosphorylation site Thr11 is inactivated.

6. The method of claim 5, wherein the MST1 antagonist is (i) a mutant PDX1 wherein the amino acid Thr11 is substituted (ii) a peptide comprising SEQ ID NO: 5 or 6 or (iii) an siRNA comprising the nucleotide sequence of any one of SEQ ID NOs: 1 to 4.

7. A pharmaceutical composition comprising a Mammalian Sterile 20-like kinase (MST) 1 antagonist.

8. The pharmaceutical composition of claim 7, further comprising at least one anti-diabetic and/or anti-obesity agent selected from the group consisting of long-acting insulin, dipeptidyl peptidase IV (DPP4) inhibitor, aldose reductase inhibitor, metformin and glucagon-like peptide (GLP).

9. The pharmaceutical composition of claim 7 formulated for oral, subcutaneous or transdermal administration.

10. (canceled)

11. A method of reducing body weight and/or enhancing the coenaesthesis in an individual comprising administering a Mammalian Sterile 20-like kinase (MST) 1 antagonist to an individual in need thereof.

12-14. (canceled)

15. A polynucleotide encoding the MST1 antagonist of claim 6.

16. A vector comprising the polynucleotide of claim 15.

17. A host cell comprising the polynucleotide of claim 15.
